(19) United States
(12) Patent Application Publication
(10) Pub. No.: US 2010/0189797 A1
(43) Pub. Date: Jul. 29, 2010

(54) ORAL ANTIDEPRESSANT FORMULATION

(76) Inventors: Julien MENDLEWICZ, Dworp (BE); Philippe Kriwin, Brussels (BE); Roland Powis De Tenbossche, Eppegem (BE)

Correspondence Address: SUGHRUE MION, PLLC, 2100 PENNSYLVANIA AVENUE, N.W., SUITE 800, WASHINGTON, DC, 20037, US

(21) Appl. No.: 12/644,041

(22) Filed: Dec. 22, 2009

Related U.S. Application Data
(63) Continuation-in-part of application No. 11/003,780, filed on Dec. 6, 2004, now Pat. No. 7,662,805.
Application No. 11/003,780 is a continuation-in-part of application No. PCT/BE03/00078, filed on Apr. 30, 2003.
Application No. PCT/BE03/00078 is a continuation-in-part of application No. PCT/BE03/00181, filed on Oct. 29, 2003.

(30) Foreign Application Priority Data
Jun. 10, 2002 (BE) PCT/BE02/00094

(51) Int. Cl. A61K 31/60; A61K 9/14

(52) U.S. Cl. 424/490; 514/159; 514/161; 514/162; 514/165

(57) ABSTRACT

Oral antidepressant formulation comprising a means for controlling the release of the pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof, with respect to the release of the compound selected from the group consisting of acetylsalicylic acid, salts and esters of acetylsalicylic acid, diaspirin, and mixtures thereof.

### RELATED APPLICATIONS

This application is a continuation in part of U.S. Ser. No. 11/003,780 filed on Dec. 6, 2004 and published on Apr. 21, 2005 under number US2005/0085450, which is a continuation in part of PCT/BE03/00078 filed on Apr. 30, 2003, published on Dec. 18, 2003 under number WO 03/103643, and claiming the priority of PCT/BE02/00094 filed on Jun. 10, 2002, as well as a continuation in part of PCT/BE03/00181 filed on Oct. 29, 2003, published on May 6, 2005 under number WO2005/039545, the disclosures of which are incorporated by reference.

### FIELD OF THE INVENTION

The present invention relates to a formulation and a method for reducing the time of onset of antidepressant action of pharmaceutically acceptable antidepressant active agents selected from the group consisting of SSRI agents, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof.

### THE PRIOR ART

Antidepressant formulations are well known. Said formulations (marketed or in development) comprise a pharmaceutically acceptable antidepressant active agent selected from the group consisting of SSRI agents, SNRIs (serotonin noradrenaline reuptake inhibitors), CRF (corticotropic releasing factor) antagonists, NK1 (neurokinin) antagonists, NK2 (neurokinin) antagonists and NK3 (neurokinin) antagonists.

The major problem of human antidepressant is the onset time required for obtaining an antidepressant action, said onset time being often greater than 4 weeks. Onset time is the time until onset of antidepressant response for a treatment with an antidepressant active agent treatment or the time to onset of therapeutic efficiency for a treatment with an antidepressant active agent. This means a great risk that the patient decides him self to stop the treatment in view of the lack of action after two weeks treatment, or to take a too large dose, possibly a toxic dose, of one or more antidepressant agents.

Another problem of antidepressant formulations is that they are inefficient for some patients (not responding patients) for reason not yet known.

### DESCRIPTION OF THE INVENTION

The present invention has for object an oral antidepressant formulation with a reduced time of onset of antidepressant action. It has been discovered that by using specific compound(s), it was possible to reduce the onset time of antidepressant action of pharmaceutically acceptable antidepressant active agents selected from the group consisting of SSRIs, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof.

The invention relates also to an oral antidepressant formulation efficient for at least 50% (preferably at least 70%, most preferably more than 80%) of the SSRI not responding patients, advantageously for at least 50% (preferably at least 70%, most preferably more than 80%) of the patients not responding to a pharmaceutically acceptable antidepressant active agent taken alone at a dose of less than 20 mg, said pharmaceutically acceptable antidepressant active agent being selected from the group consisting of SNRIs (serotonin noradrenaline reuptake inhibitors), CRF (corticotropic releasing factor) antagonists, NK1 (neurokinin) antagonists, NK2 (neurokinin) antagonists and NK3 (neurokinin) antagonists. It has been discovered that by using specific compound(s), it was possible to render therapeutically active pharmaceutically acceptable antidepressant active agents selected from the group consisting of SSRIs, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof, for patients or humans not responding to antidepressant taken alone, such as SSRI taken alone.

By antidepressant agent, it is meant in the present specification the antidepressant agent as in its free base form as well as in a pharmaceutically acceptable acid addition salt form (for example salt of (±)-N-methyl-3-phenyl-3-[(α-α-α-trifluoro-p-tolyl)oxy] propylamine in case of fluoxetine).

Such acid addition salts include salts derived from inorganic acids such as: hydrochloric acid, nitric acid, phosphoric acid, sulfuric acid, hydrobromic acid, hydriodic acid, nitrous acid, phosphorous acid and the like, as well as salts of non-toxic organic acids including aliphatic mono- and dicarboxylates, phenyl-substituted alkanoates, hydroxy alkanoates and alkanedioates, aromatic acids, aliphatic and aromatic sulfonic acids, etc. Such pharmaceutically-acceptable salts thus include: sulfate, pyrosulfate, bisulfate, sulfite, bisulfite, nitrate, phosphate, monohydrogenphosphate, dihydrogenphosphate, metaphosphate, pyrophosphate, chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, benzenesulfonates, toluenesulfonate, chlorobenzenesulfonate, xylenesulfonate, phenylacetate, phenylpropionate, phenylbutyrate, citrate, lactate, 8-hydroxybutyrate, glycolate, malate, tartrate, methanesulfonate, propanesulfonates, naphthalene-1-sulfonate, naphthalene-2-sulfonate, mandelate and the like salts.

The invention relates to an oral antidepressant formulation comprising an effective antidepressant amount of one or more pharmaceutically acceptable antidepressant active agents selected from the group consisting of selective serotonin reuptake inhibitors (SSRI), SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof (most preferably SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof), and an effective amount of at least a compound or additive selected from the group consisting of acetylsalicylic acid (ASA), salts thereof, ester thereof, the acid form being however preferred, diaspirin and mixtures thereof, for reducing the onset time of antidepressant action of the pharmaceutically acceptable antidepressant active agents selected from the group consisting of selective serotonin reuptake inhibitor(s), SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof. ASA, salts and esters thereof, and mixtures thereof are however the preferred compound(s) to be combined with the pharmaceutically acceptable antidepressant active agents selected from the group consisting of SSRIs, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof.

Advantageously, the amount of aspirin, diaspirin or salts and esters of acetylsalicylic acid, and combinations thereof is lower than 50 mg, preferably lower than 40 mg, such as comprised between 5 and 35 mg.

While the mechanism of action is not clear, it seems that the reduced onset or the efficiency of the antidepressant for patient (human) not responding to SSRI taken alone at dose of less than 20 mg is due to one or more of the following reasons:
better passage of the antidepressant active agent in the blood
higher fluidity of the blood
increased deposit in the brain
activation of the brain or specific sites thereof, so that the brain or some specific sites have a higher receptivity for the antidepressant active agent

One or more other advantages of the formulation of the invention are:
quicker action
less headaches
possibility to reduce the doses of antidepressant
less risk factor for suicide (for example due to the immediate action of the antidepressant)
efficient for SSRI not responding patients
etc.

The formulation comprises advantageously an effective amount of at least a compound selected from the group consisting of acetylsalicylic acid, salts and esters of acetylsalicylic acid, and mixtures thereof, for reducing at least 15%, advantageously at least 20%, preferably at least 50% the average onset of action of the pharmaceutically acceptable antidepressant active agents selected from the group consisting of SSRI, SNRIs (serotonin noradrenaline reuptake inhibitors), CRF (corticotropic releasing factor) antagonists, NK1 antagonists, NK2 antagonists, NK3 antagonists and combinations thereof, most preferably from the group consisting of SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof. Preferably, the effective amount of one or more compounds (selected from the group consisting of aspirin, salts thereof, esters thereof, diaspirin and combinations thereof) for reducing the onset time of action of the pharmaceutically acceptable antidepressant active agents selected from the group consisting of SSRI, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof is lower than 5 times the amount of pharmaceutically acceptable antidepressant active agents selected from the group consisting of SSRI, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof.

Most preferably, the effective amount of one or more compounds (selected from the group consisting of aspirin, salts thereof, esters thereof, diaspirin and combinations thereof) for reducing the onset time of action of pharmaceutically acceptable antidepressant active agents selected from the group consisting of SSRI, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof is lower than 2 times, advantageously lower than 1.5 times, preferably lower than 1 times the amount of pharmaceutically acceptable antidepressant active agents selected from the group consisting of SSRI, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof. According to a preferred embodiment, the formulation comprises a quick release effective amount of at least a compound selected from the group consisting of acetylsalicylic acid, salts and esters of acetylsalicylic acid, diaspirin, and mixtures thereof, for reducing the onset of action of the selective serotonin reuptake inhibitor(s).

According to a detail of a specific embodiment, the formulation comprises particles, core, microparticles, spherical particles comprising at least one pharmaceutically acceptable antidepressant active agent selected from the group consisting of SSRIs selective serotonin reuptake inhibitors, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof, and possibly at least a compound selected from the group consisting of acetylsalicylic acid, salts and esters of acetylsalicylic acid, and mixtures thereof, said particles, core, microparticles, spherical particles being advantageously provided with a coating comprising at least a compound selected from the group consisting of acetylsalicylic acid, salts and esters of acetylsalicylic acid, diaspirin, and mixtures thereof, for reducing the onset time of action of the pharmaceutically acceptable antidepressant active agents selected from the group consisting of SSRIs selective serotonin reuptake inhibitors, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof, said coating being substantially free of pharmaceutically acceptable antidepressant active agents selected from the group consisting of SSRIs selective serotonin reuptake inhibitors, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof. The coating is for example made by using a water-soluble polymer. It has however to be noted that in case the SSRI and the additive selected from the group consisting of acetylsalicylic acid, salts and esters of acetylsalicylic acid, and mixtures thereof, are mixed together so as to prepare matrix comprising both compounds, the additive acts as means for ensuring a better dissolution of the pharmaceutically acceptable antidepressant active agents selected from the group consisting of SSRI, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof, possibly mixed with SSRI and/or a better passage in the organism. It can be advantageous to provide a barrier coating between the SSRI containing core, particles, etc. and the coating containing the additive, so as to avoid any interaction between the pharmaceutically acceptable antidepressant active agents selected from the group consisting of SSRIs selective serotonin reuptake inhibitors, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof, and the additive. Such a barrier coating is for example a water insoluble coating (polymer, etc.). This is advantageous for avoiding any stability problem.

Advantageously, the composition of the invention comprises a pharmaceutically acceptable antidepressant active agent selected from the group consisting of SSRIs selective serotonin reuptake inhibitors, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof, the SSRI (selective serotonin reuptake inhibitor) being most preferably selected from the group consisting of citalopram, especially escitalopram or the s-isomer of citalopram, paroxetine, sertraline, fluvoxamine, fluoxetine. Fluoxetine, citalopram, escitalopram and sertraline are the most preferred SSRI.

While the formulation can be liquid, the formulation is advantageously solid. Solid formulation means a formulation which has a solid aspect, even if the pharmaceutically acceptable antidepressant active agent selected from the group consisting of SSRIs selective serotonin reuptake inhibitors, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof is in a liquid or semi liquid form in a carrier or on a carrier. For example, the solid form comprises from 4 mg up to 100 mg, preferably from 4 to 20 mg of pharmaceutically acceptable antidepressant active agents selected from the group consisting of SSRIs selective serotonin reuptake inhibitors, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof.

The invention relates further to an antidepressant system comprising at least a formulation, advantageously an oral formulation, with an effective antidepressant amount of one or more pharmaceutically acceptable antidepressant active agents selected from the group consisting of SSRIs selective serotonin reuptake inhibitors, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof, and at least a formulation, advantageously an oral formulation, with an effective amount of at least a compound selected from the group consisting of acetylsalicylic acid, salts and esters of acetylsalicylic acid, diaspirin, and mixtures thereof, for reducing the onset of action of pharmaceutically acceptable antidepressant active agents selected from the group consisting of SSRIs selective serotonin reuptake inhibitors, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof. The two formulations can be administered simultaneously or successively, and/or with different administration pathways (oral, rectal, parenteral, etc.) or with the same administration pathways. The system of the invention has advantageously one or more characteristics of the oral formulation of the invention as described here above.

The invention relates also to an oral antidepressant formulation for human comprising an effective antidepressant amount of at least one pharmaceutically acceptable antidepressant active agent selected from the group consisting of SSRI agents, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof, and an effective amount of at least one compound selected from the group consisting of acetylsalicylic acid, salts and esters of acetylsalicylic acid, diaspirin, and mixtures thereof, for reducing the onset time of action of the pharmaceutically acceptable antidepressant active agent selected from the group consisting of SSRI agents, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof, whereby said formulation comprises a means for controlling the release of the pharmaceutically acceptable antidepressant active agent selected from the group consisting of SSRI agents, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof, with respect to the release of the compound selected from the group consisting of acetylsalicylic acid, salts and esters of acetylsalicylic acid, diaspirin, and mixtures thereof. Advantageously, the formulation comprises a means for delaying the start of release of the pharmaceutically acceptable antidepressant active agent selected from the group consisting of SSRI agents, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof, from 3 minutes to 60 minutes, preferably from 5 minutes to 45 minutes, most preferably from 7 minutes to 30 minutes, with respect to the release of at least 50% by weight (advantageously at least 75% by weight, preferably at least 85% by weight) of the compound selected from the group consisting of acetylsalicylic acid, salts and esters of acetylsalicylic acid, diaspirin, and mixtures thereof.

According to a preferred embodiment, the formulation comprises a form containing the pharmaceutically acceptable antidepressant active agent selected from the group consisting of SSRI agents, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof, said form being provided with a coating for delaying the start of the release of the antidepressant agent with respect to the release of at least 50% by weight (advantageously at least 75% by weight, preferably at least 85% by weight) of the compound selected from the group consisting of acetylsalicylic acid, salts and esters of acetylsalicylic acid, diaspirin, and mixtures thereof. Advantageously, the form is provided with a coating for delaying from 3 minutes to 60 minutes, preferably from 5 minutes to 45 minutes, most preferably from 7 minutes to 30 minutes the start of the release of the antidepressant agent with respect to the release of at least 50% by weight (advantageously at least 75% by weight, preferably at least 85% by weight) of the compound selected from the group consisting of acetylsalicylic acid, salts and esters of acetylsalicylic acid, diaspirin, and mixtures thereof.

The invention relates also to a method of treatment of a patient suffering a depression (especially patient known as resistant to SSRI at a dose of 20 mg for a patient weight of 70 kg or not responding to SSRI at a dose of 20 mg for a patient dose of 70 kg), in which the patient is administered an effective antidepressant amount of one or more pharmaceutically acceptable antidepressant active agents selected from the group consisting of SSRIs selective serotonin reuptake inhibitors, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof, such as mixtures of one or more selective serotonin reuptake inhibitors with one or more CRF (corticotropic releasing factor) antagonists and/or NK1 (neurokinin) antagonists and/or NK2 (neurokinin) antagonists and/or NK3 (neurokinin) antagonists, and an effective amount of at least an additive compound selected from the group consisting of acetylsalicylic acid, salts and esters of acetylsalicylic acid, diaspirin, and mixtures thereof, for reducing the onset time of antidepressant action of the pharmaceutically acceptable antidepressant agent selected from the group consisting of SSRIs (selective serotonin reuptake inhibitors), SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof. The amount of additive (acetylsalicylic acid, salts and esters of acetylsalicylic acid, diaspirin, and mixtures thereof) is lower than 50 mg for a human with a weight of 70 kg, preferably comprised between 5 mg and 40 mg. In said method, a formulation of the invention is preferably used. However, the method of the invention can be carried out by other means. For example, the pharmaceutically acceptable antidepressant active agents selected from the group consisting of SSRIs (selective serotonin reuptake inhibitors), SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof and the additive compound are administered successively, preferably first the additive is administered and then the pharmaceutically acceptable antidepressant active agents selected from the group consisting of SSRIs, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof. The pharmaceutically acceptable antidepressant active agents selected from the group consisting of SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof and the additive can be administered by different way, for example the pharmaceutically acceptable antidepressant active agents selected from the group consisting of SSRIs, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof orally, while the additive through the skin or subcutaneous injection (for example in a vein).

In the method of the invention, the doses of pharmaceutically acceptable antidepressant active agents advantageously selected from the group consisting of SSRIs, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof to be administered to the patient are for example the doses proposed in the commercial formulation of pharmaceutically acceptable antidepressant active agents selected from the group consisting of SSRIs, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof or by the doctors with such commercial formulation. However, these doses can possibly be lowered, for example by a factor 2 or 3.

As it seems also that the time of action (or time efficiency) of the pharmaceutically acceptable antidepressant active agents advantageously selected from the group consisting of SSRIs, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof is increased by the additive, less doses have to be taken with respect to the commercial formulations. For example one daily dose with the formulation of the invention is sufficient, while two or more doses were required for a commercial SSRI formulation, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof.

When using oral dosage form in the system, formulation or methods of the invention, it seems to be advantageous to release the compound selected from the group consisting of ASA, salts thereof, esters thereof, diaspirin and mixtures thereof prior to the release of the antidepressant agent. It seems preferable that the compound selected from the group consisting of ASA, salts thereof, esters thereof, diaspirin and mixtures thereof is released (preferably substantially completely) from 3 minutes to 60 minutes (advantageously from 5 minutes to 45 minutes, preferably from 7 minutes to 30 minutes) prior to the release of the antidepressant agent. For controlling the release of one compound with respect to the other compound, the antidepressant compound is placed in a form, solid form provided with a control means, such as an outer coating or barrier, such as a thick polyacrylate layer, while the ASA is in a quick release form.

The delayed release of the antidepressant agent with respect to the release of the compound selected from the group consisting of ASA, salts and esters thereof, diaspirin, and mixtures thereof can be determined by placing the oral combined form in a aqueous medium having a pH of 1.2 (900 ml), said medium maintained at 37° C. being agitated with paddles apparatus at 100 rpm. The release of the antidepressant agent can be determined and compared with respect to the release of the compound selected from the group consisting of ASA, salts and esters thereof, diaspirin, and mixtures thereof. The start of the release of the antidepressant agent is considered as being as the moment when 5% by weight of the antidepressant agent is released in the medium.

The delayed action or time can also be determined by separating the antidepressant agent (for example in the form of coated beads) from the compound selected from the group consisting of ASA, salts and esters thereof, diaspirin, and mixtures thereof. The antidepressant form free of the compound selected from the group consisting of ASA, salts and esters thereof, diaspirin, and mixtures thereof is tested in order to determine the time T1 required for achieving a 5% by weight release. The compound selected from the group consisting of ASA, salts and esters thereof, diaspirin, and mixtures thereof is tested in order to determine the moment or time T2, T2′, T2″ when 50% by weight, 75% by weight and 85% by weight is released. By making the difference between T1 and respectively T2, T2′ and T2″, it is possible to determine the delayed time of the antidepressant with respect to the release of respectively 50%, 75% and 85% of the compound selected from the group consisting of ASA, salts and esters thereof, diaspirin, and mixtures thereof.

In the method of the invention, the start of the release of the pharmaceutically acceptable antidepressant active agent selected from the group consisting of SSRI agents, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof, is advantageously delayed from 3 minutes to 60 minutes (preferably from 5 minutes to 45 minutes, most preferably from 7 minutes to 30 minutes) with respect to the release of at least 50% by weight (advantageously at least 75% by weight, preferably at least 85% by weight) of the effective amount of the compound selected from the group consisting of acetylsalicylic acid, salts and esters of acetylsalicylic acid, diaspirin, and mixtures thereof.

According to a further embodiment of the method of the invention, the pharmaceutically acceptable antidepressant active agent selected from the group consisting of SSRI agents, SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof, is administered from 1 minute to 60 minutes (advantageously from 3 minutes to 60 minutes, preferably from 5 minutes to 45 minutes) after the administration of the effective amount of the compound selected from the group consisting of acetylsalicylic acid, salts and esters of acetylsalicylic acid, diaspirin, and mixtures thereof.

The pharmaceutically acceptable antidepressant active agents selected from the group consisting of SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof are advantageously compounds as disclosed in the following references: for SNRIs (serotonin noradrenaline reuptake inhibitors), CRF (corticotropic releasing factor) antagonists: U.S. Pat. No. 6,525,067; U.S. Pat. No. 6,432,989; U.S. Pat. No. 6,613,777; U.S. Pat. No. 6,610,678; U.S. Pat. No. 6,589,958; U.S. Pat. No. 6,589,952; U.S. Pat. No. 6,469,166,

for NK1 (neurokinin) antagonists: U.S. Pat. No. 6,635,630, U.S. Pat. No. 6,096,766
for NK2 (neurokinin) antagonists: U.S. Pat. No. 5,889,024; U.S. Pat. No. 6,444,809, U.S. Pat. No. 6,355,695, U.S. Pat. No. 6,124,279; U.S. Pat. No. 6,008,223; U.S. Pat. No. 5,977,135; U.S. Pat. No. 5,567,700; U.S. Pat. No. 5,521,199,
for NK3 (neurokinin) antagonists: U.S. Pat. No. 6,040,316; U.S. Pat. No. 6,602,886; U.S. Pat. No. 6,348,330; U.S. Pat. No. 6,258,943,
for combination of NK1 (neurokinin) antagonist with NK2 antagonist: U.S. Pat. No. 6,177,450, U.S. Pat. No. 6,147,083,

Examples of SNRIs are commercial products such as Venlafaxine, duloxetine, milnacipram, reboxetine, etc., and mixtures thereof.

The invention relates advantageously to a unit dosage form suitable for oral administration to a human comprising:
a first SSRI containing form comprising an effective antidepressant amount of a SSRI compound selected from the group consisting of fluoxetine, citalopram, escitalopram, paroxetine, venlafaxine, duloxetine, dapoxetine, fluvoxamine, sertraline, and pharmaceutically acceptable salts thereof, at least one pharmaceutically acceptable carrier,
at least one release delay means for the SSRI compound in an amount adapted for ensuring at least a delay release period selected from the group consisting of a delay release period at a pH between 1 and 4.5 of at least 20 minutes with a rate of release of less than 2 mg SSRI per hour, preferably less than 1 mg/hour, a delay release period being the sum of a first delay release period at pH comprised between 1 and 4.5 of at least 20 minutes, advantageously at least 60 minutes, such as at least 120 minutes, with a rate of release of less than 2 mg (advantageously less than 1 mg) SSRI per hour and of a second delay release period at pH comprised between 6.8 and 7.5 of at least 10 minutes, advantageously at least 20 minutes, for example comprised 20 minutes and 60 minutes, with a rate of release of less than 2 mg SSRI per hour, preferably less than 1 mg SSRI/hour, and
a second form comprising an effective amount of less than 50 mg of an additional active agent selected from the group consisting of acetyl salicylic acid, pharmaceutically acceptable salts thereof, pharmaceutically acceptable esters thereof, diaspirin and mixtures thereof,
whereby said unit dosage form is adapted so that at least 90% (preferably more than 95% or even substantially 100%) by weight of said additional active agent of the second form is released within the said delay release period for the SSRI compound selected from the group consisting of a delay release period at a pH between 1 and 4.5 of at least 20 minutes, advantageously at least 60 minutes, such as at least 120 minutes, with a rate of release of less than 2 mg SSRI per hour, advantageously less than 1 mg SSRI per hour, a delay release period being the sum of a first delay release period at pH comprised between 1 and 4.5 of at least 20 minutes, advantageously at least 60 minutes, such as at least 120 minutes, with a rate of release of less than 2 mg SSRI per hour, advantageously less than 1 mg SSRI per hour, and of a second delay release period at pH comprised between 6.8 and 7.5 of at least 10 minutes, advantageously at least 20 minutes, for example comprised between 20 minutes and 60 minutes, with a rate of release of less than 2 mg SSRI per hour, preferably less than 1 mg SSRI/hour.

After said delay period, the SSRI agent is released, for example at least partly at a rate of more than 2 mg SSRI/hour, advantageously more than 5 mg/hour, such as from 5 to 50 mg SSRI released within the first hour following the end the release delay period.

The unit dosage form is adapted for ensuring a differentiate release of the SSRI with respect to the additional active agent, i.e. for enabling first the release of the additional active agent in the stomach and/or in the duodenum and/or in the small intestine (preferably in the duodenum (first portion of the small intestine following the stomach) and/or intestine, most preferably in the small intestine), and then the release of the SSRI, preferably only in the duodenum and/or the intestine, preferably only in the intestine. The release of the additional active agent in the stomach or preferably in the first portion of the small intestine following the stomach is advantageously a substantially immediate release, while the release of the SSRI advantageously within the intestine, such as small intestine can be an immediate release form or a sustained release form.

Such a time differentiated release between the release of the additional active agent and the release of the SSRI is very advantageous for preventing possible increased side effects due to the combined release of additional active agent or ASA and SSRI, as well for preventing possible degradation of the SSRI by the additional active agent.

The unit dosage form of the invention is thus advantageously a kind spatially controlled release form, as the additional active agent is first released, while the SSRI being released after the substantially complete release of the additional active agent, salts or esters thereof. The release of the additional active agent is advantageously quite immediate after the administration of the oral unit dosage form or in the stomach or in the first portion of the intestine, especially small intestine, while the delayed SSRI release can be an immediate and/or sustained release after the elapse of a time period from the start of the release of the additional active agent, for example after the elapse of a period from 3 to 60 minutes from the start of the release of the additional active agent, preferably 5 to 15 minutes after the release of at least 90% by weight of the additional active agent.

According to an embodiment, the unit dosage form comprises:
a first SSRI containing form comprising an effective antidepressant amount of a SSRI compound selected from the group consisting of fluoxetine, citalopram, escitalopram, paroxetine, venlafaxine, duloxetine, dapoxetine, fluvoxamine, sertraline, and pharmaceutically acceptable salts thereof, at least one pharmaceutically acceptable carrier, and at least one release delay means in an amount adapted for ensuring at least a delay release period selected from the group consisting of a delay release period at a pH between 1 and 4.5 of at least 20 minutes, advantageously at least 60 minutes, for example 120 minutes or more, with a rate of release of less than 2 mg SSRI per hour, advantageously less than 1 mg SSRI per hour, and a delay release period being the sum of a first delay release period at pH comprised between 1 and 4.5 of at least 20 minutes, advantageously at least 60 minutes, for example 120 minutes or even more, with a rate of release of less than 2 mg SSRI per hour, advantageously less than 1 mg SSRI per hour, and of a second delay release period at pH comprised between 6.8 and 7.5 of at least 10 minutes, advantageously at least 20 minutes, for example comprised between 20 minutes and 60 minutes, with a rate of release of less than 2 mg SSRI per hour, advantageously less than 1 mg SSRI per hour, and
a second form comprising an effective amount of less than 50 mg of an additional active agent selected from the group consisting of acetyl salicylic acid, pharmaceutically acceptable salts thereof, pharmaceutically acceptable esters thereof, diaspirin and mixtures thereof, whereby said form is adapted so that at least 90% by weight of said additional active agent is released within the said delay release period for the SSRI compound selected from the group consisting of a delay release period at a pH between 1 and 4.5 of at least 20 minutes, advantageously at least 60 minutes, such as 120 minutes or even more, with a rate of release of less than 2 mg SSRI per hour, advantageously less than 1 mg/hour, a delay release period being the sum of a first delay release period at pH comprised between 1 and 4.5 of at least 20 minutes, advantageously at least 60 minutes, for example 120 minutes or more, with a rate of release of less than 2 mg SSRI per hour, advantageously less than 1 mg/hour, and of a second delay release period at pH comprised between 6.8 and 7.5 of at least 10 minutes, advantageously at least 20 minutes, comprised between 20 minutes and 60 minutes, with a rate of release of less than 2 mg SSRI per hour, advantageously less than 1 mg SSRI per hour.

According to a further embodiment, the unit dosage form comprises an envelope containing at least the first SSRI containing form and the second form containing the additional active agent, whereby the envelope comprises at least one enteric coating, said envelope being adapted for preventing substantially any release of the SSRI compound and the additional active agent in the stomach (i.e. in an acid medium with a pH comprised between 1 and 4.5), said envelope being selected from the group consisting of envelope rendered permeable at pH comprised between 6 and 7.5, envelope disintegrable (partly or completely, for example breakable) at pH comprised between 6 and 7.5, envelope solubilized at pH comprised between 6 and 7.5, and combinations thereof and whereby the first SSRI containing form, advantageously in the form of one or more pellets, is provided with a delay release means, advantageously coating, for ensuring a delay release period of at least 10 minutes, advantageously of at least 20 minutes, for example comprised between 20 minutes and 60 minutes, for the SSRI compound at pH comprised between 6.8 and 7.5 with a rate of release of less than 2 mg SSRI per hour, advantageously less than 1 mg SSRI/hour.

The envelope can be a capsule provided with one enteric coating. In the present specification enteric coating means one or more layers ensuring an enteric barrier, for preventing the envelope or capsule to be rendered permeable in the stomach, to be solubilized within the stomach, and to be degraded with the stomach.

Enteric capsule can be prepared by using the Enteric capsule coater marketed by Feton International SA, Belgium. When coating a capsule with the enteric polymer formulation, the enteric coating surrounds the envelope.

Advantageously, the second form containing the additional active agent is a form adapted for ensuring the release of at least 90% of said additional active agent in less than 5 minutes at a pH comprised between 6 and 7.5, i.e. for ensuring the release of said additional active agent within the first portion of the intestine.

According to a further embodiment, the unit dosage form comprises a water soluble envelope containing at least the first SSRI containing form and the second form containing the additional active agent, whereby the first SSRI containing form is provided with a delay release means, advantageously an enteric coating, adapted for preventing substantially any SSRI release in the stomach, while ensuring a delay release period of at least 10 minutes, advantageously at least 20 minutes, for example comprised between 20 minutes and 60 minutes, for the SSRI compound at pH comprised between 6.8 and 7.5 with a rate of release of less than 2 mg SSRI per hour, advantageously less than 1 mg.

Advantageously, the second form containing the additional active agent is a form adapted for ensuring the release of at least 90% of said additional active agent in less than 5 minutes at a pH comprised between 6 and 7.5. The second form is preferably also provided with an enteric coating, so as to prevent or reduce the release of said additional active agent in the stomach. The second form is for example in the form of pellets containing the additional active agent.

The second form containing the additional active agent is advantageously a form adapted for preventing substantially any release of the additional active agent in the stomach, while ensuring the release of at least 90% of said additional active agent in less than 5 minutes at a pH comprised between 6 and 7.5. For example, the second form is in the form of pellets containing the additional active agent, whereby said pellets are provided with an enteric coating or coating layer adapted for preventing substantially any release of the additional active agent in the stomach, while ensuring the release of at least 90% of said additional active agent in less than 5 minutes at a pH comprised between 6 and 7.5. The second form is thus advantageously a form enabling a substantially immediate release of the additional active agent when being in the intestine, especially in the small intestine.

According to a specific embodiment, the first SSRI containing form is a SSRI containing form (such as a core, granules, particles, tablets, matrix, etc) provided with a coating comprising at least an inwards coating layer, an outwards coating layer, and an intermediate coating layer located between the inwards coating layer and the outwards coating layer, whereby the inwards coating layer is located between the SSRI containing core and the outwards coating layer, whereby the intermediate coating layer comprises the additional active agent, while the inwards coating layer is an enteric coating layer adapted for preventing substantially any SSRI release in the stomach, while ensuring a delay release period of at least 10 minutes, advantageously at least 20 minutes, for example comprised between 20 minutes and 60 minutes for the SSRI compound at pH comprised between 6.8 and 7.5 with a rate of release of less than 2 mg SSRI per hour, advantageously less than 1 mg SSRI/hour.

With respect to the SSRI containing form, the inwards coating layer is more adjacent to the form than the outwards coating layer. The coating can comprise one or more additional coating layers, such as one or more protective layers.

According to a further specific embodiment, the first SSRI containing form is a SSRI containing form (such as a core, granules, pellets, tablets, matrix, etc) provided with a surrounding coating comprising at least an inwards surrounding coating layer, an outwards surrounding coating layer, and an intermediate coating layer located between the inwards coating layer and the outwards coating layer, whereby the inwards surrounding coating layer is located between the SSRI containing core and the outwards surrounding coating layer, whereby the intermediate coating layer comprises the additional active agent, while the outwards surrounding coating layer is an enteric coating layer adapted for preventing substantially any SSRI release and any additional active agent release in the stomach, and while the inwards coating layer is adapted for ensuring a delay release period of at least 10 minutes, advantageously at least 20 minutes, for example comprised between 20 minutes and 60 minutes, for the SSRI compound at pH comprised between 6.8 and 7.5 with a rate of release of less than 2 mg SSRI per hour, advantageously less than 1 mg SSRI per hour.

Still according to a further embodiment, the unit dosage form suitable for oral administration to a human, comprising:
a first SSRI containing form comprising an effective antidepressant amount of a SSRI compound selected from the group consisting of fluoxetine, citalopram, escitalopram, paroxetine, venlafaxine, duloxetine, dapoxetine, fluvoxamine, sertraline, and pharmaceutically acceptable salts thereof, at least one pharmaceutically acceptable carrier, and at least one release delay means in an amount adapted, so that, when dissolution tested in vitro in a USP-2 apparatus containing 900 ml of acetate buffer, pH 4.0, which is 0.075 M in NaCl, said USP-2 apparatus being equipped with a paddle stirring at 50 rpm, the SSRI containing form releases the SSRI compound at a rate less than 1 mg SSRI/hr for a delay period comprised between 10 minutes and 4 hours, advantageously between 20 minutes and 4 hours, for example between 20 minutes and 1 hour and, thereafter at a rate greater than 1 mg SSRI/hr, advantageously at a rate from 1 mg SSRI/hr to 40 mg SSRI/hr for a period of at least 1 hour after the delay period, for example for at least two hours, and
a second form comprising an effective amount of less than 50 mg of an additional active agent selected from the group consisting of acetyl salicylic acid, pharmaceutically acceptable salts thereof, pharmaceutically acceptable esters thereof, diaspirin and mixtures thereof, whereby said form is adapted, when dissolution tested in vitro in a USP-2 apparatus containing 900 ml of acetate buffer, pH 4.0, which is 0.075 M in NaCl, releases at least 90% by weight of the additional active agent in less than the said delay period.

According to an embodiment, the first SSRI containing form comprises an effective antidepressant amount of a SSRI compound selected from the group consisting of fluoxetine, citalopram, escitalopram, paroxetine, venlafaxine, duloxetine, dapoxetine, fluvoxamine, sertraline, and pharmaceutically acceptable salts thereof, at least one pharmaceutically acceptable carrier, at least one release delay means, and at least a control sustain release means, whereby said release delay means and control sustain release means are present in an amount adapted, so that, when dissolution tested in vitro in a USP-2 apparatus containing 900 ml of acetate buffer, pH 4.0, which is 0.075 M in NaCl, said USP-2 apparatus being equipped with a paddle stirring at 50 rpm, the SSRI containing form releases the SSRI compound at a rate less than 1 mg SSRI/hr for a delay period comprised between 20 minutes and 2 hours and, thereafter at a rate from 1 mg SSRI/hr to 40 mg SSRI/hr for a period of at least 1 hour after the delay period, advantageously provided that less than 80% by weight of the SSRI compound is released within said period of 1 hour.

For example, the first SSRI containing form comprises a core element selected from the group consisting of matrix tablet, osmotic tablet, hydrogel tablet, multiparticulates and combinations thereof, said core element being provided with an outer coating comprising at least one release delay layer, said outer coating being adapted, so that, when dissolution tested in vitro in a USP-2 apparatus containing 900 ml of acetate buffer, pH 4.0, which is 0.075 M in NaCl, said USP-2 apparatus being equipped with a paddle stirring at 50 rpm, the SSRI containing form releases the SSRI compound at a rate less than 1 mg SSRI/hr for a delay period comprised between 20 minutes and 2 hours and, thereafter at a rate greater than 1 mg SSRI/hr which remains substantially intact during the period of sustained release.

According to a further example, the first SSRI containing form comprises a core element selected from the group consisting of matrix tablet, osmotic tablet, hydrogel tablet, multiparticulates and combinations thereof, said core element being placed within a capsule adapted, so that, when dissolution tested in vitro in a USP-2 apparatus containing 900 ml of acetate buffer, pH 4.0, which is 0.075 M in NaCl, said USP-2 apparatus being equipped with a paddle stirring at 50 rpm, the SSRI containing form releases the SSRI compound at a rate less than 1 mg SSRI/hr for a delay period comprised between 20 minutes and 2 hours and, thereafter at a rate greater than 1 mg SSRI/hr which remains substantially intact during the period of sustained release.

The second form comprising an effective amount of less than 50 mg of an additional active agent selected from the group consisting of acetyl salicylic acid, pharmaceutically acceptable salts thereof, pharmaceutically acceptable esters thereof, diaspirin and mixtures thereof, is advantageously in the form selected from the group consisting of tablets, microtablets, multiparticulates, powders, pellets, capsule and combinations thereof.

The second form of any embodiments, possibly in the form of a coating layer, advantageously further comprises at least a water-soluble pharmaceutically acceptable carrier having a water solubility at 37° C. of more than 5 g per 100 ml water. This is advantageous for increasing the release of the additional active agent.

Preferably, the second form comprises an effective amount of less than 50 mg of an additional active agent selected from the group consisting of acetyl salicylic acid, pharmaceutically acceptable salts thereof, pharmaceutically acceptable esters thereof, diaspirin and mixtures thereof, and a water soluble pharmaceutically acceptable carrier having a water solubility at 37° C. of more than 25 g per 100 ml water.

The water-soluble pharmaceutically acceptable carrier is more preferably selected from the group consisting of saccharides and glycine betaine and salts thereof.

According to an embodiment, the unit dosage form comprises less than 300 mg glycine betaine or salt thereof, for example between 100 mg and 250 mg. Said betaine is preferably present in the second form, while being possibly also present in the first form.

The glycine betaine carrier, granule or beads is suitable for preparing pharmaceutical pellets comprising one or more pharmaceutically active agents. Said glycine betaine beads, granules have advantageously a diameter lower than 3 mm and greater than 250 μm, and are provided with one or more coating layers.

In the unit dosage form of the invention, the first SSRI containing form is selected from the group consisting of solid form, semi solid form and combinations thereof, while the second form is selected from the group consisting of solid form, semi solid form and combinations thereof.

According to a further possible embodiment, the unit dosage form comprises a capsule selected from the group consisting of water soluble capsule, breakable capsule at pH lower than 4, breakable capsule at pH comprised between 7 and 8, said capsule containing:
a first SSRI containing solid form comprising an effective antidepressant amount of a SSRI compound selected from the group consisting of fluoxetine, citalopram, escitalopram, paroxetine, venlafaxine, duloxetine, dapoxetine, fluvoxamine, sertraline, and pharmaceutically acceptable salts thereof, said form having a shape selected from the group consisting of tablets provided with a surrounding or outer coating, multiparticulate provided with an outer or surrounding coating, whereby said outer or surrounding coating comprises at least an polymer layer resistant to acid medium with a pH below 4, while being rendered permeable at pH comprised between 7 and 8, and
a second solid form comprising an effective amount of less than 50 mg of an additional active agent selected from the group consisting of acetyl salicylic acid, pharmaceutically acceptable salts thereof,
pharmaceutically acceptable esters thereof, diaspirin and mixtures thereof, whereby said form is adapted, when dissolution tested in vitro in a USP-2 apparatus containing 900 ml of acetate buffer, pH 4.0, which is 0.075 M in NaCl, releases at least 90% by weight of the additional active agent in less than the said delay period.

According to still an embodiment, the first SSRI containing form comprises a core element selected from the group consisting of matrix tablet, osmotic tablet, hydrogel tablet, multiparticulates and combinations thereof, said core element being provided with an outer or surrounding coating comprising at least one release delay layer, said surrounding or outer coating being adapted, so that, when dissolution tested in vitro in a USP-2 apparatus containing 900 ml of acetate buffer, pH 4.0, which is 0.075 M in NaCl, said USP-2 apparatus being equipped with a paddle stirring at 50 rpm, the SSRI containing form releases the SSRI compound at a rate less than 1 mg SSRI/hr for a delay period comprised between 15 minutes and 2 hours and, thereafter at a rate greater than 1 mg SSRI/hr, whereby said outer coating comprises at least a polymer layer ensuring a sustained release, said polymer layer preventing the release of SSRI through the layer at the pH of the stomach, while being permeable at the pH of the small intestine.

According to a still further embodiment, the unit dosage form comprises a capsule selected from the group consisting of water soluble capsule and breakable capsule at pH lower than 4, said capsule containing:
a first SSRI containing solid form comprising an effective antidepressant amount of a SSRI compound selected from the group consisting of fluoxetine, citalopram, escitalopram, paroxetine, venlafaxine, duloxetine, dapoxetine, fluvoxamine, sertraline, and pharmaceutically acceptable salts thereof, at least one pharmaceutically acceptable carrier, and at least one outer or surrounding coating layer comprising at least one release delay layer, so that, when dissolution tested in vitro in a USP-2 apparatus containing 900 ml of acetate buffer, pH 4.0, which is 0.075 M in NaCl, said USP-2 apparatus being equipped with a paddle stirring at 50 rpm, the SSRI containing form releases the SSRI compound at a rate less than 1 mg SSRI/hr for a delay period comprised between 20 minutes and 1 hour and, thereafter at a rate greater than 1 mg SSRI/hr, and
a second solid form comprising an effective amount of less than 50 mg of an additional active agents selected from the group consisting of acetyl salicylic acid, pharmaceutically acceptable salts thereof, pharmaceutically acceptable esters thereof, diaspirin and mixtures thereof, whereby said form is adapted, when dissolution tested in vitro in a USP-2 apparatus containing 900 ml of acetate buffer, pH 4.0, which is 0.075 M in NaCl, releases at least 90% by weight of the additional active agent in less than the said delay period.

In order to delay the release of SSRI, with respect to the time of release of the additional active agent, use can be done of an outwards or surrounding coating useful for delaying the release of SSRI once the unit dosage form is ingested, said outwards coating breaks down and becomes permeable once the tablet has left the stomach or after a preset time, for example when the first SSRI containing form is already for at least 10 minutes within the small intestine, i.e. in a medium having a pH comprised between 6 and 7.5, more specifically between 6.8 and 7.5. Possibly, the outwards coating surrounds an inwards coating, the latter being present, so as to control the release of SSRI, so as to achieve a sustained release. However, in embodiments, after the outwards layer being permeable or broken, the SSRI release can be substantially immediate in the intestine.

Materials useful as delay release means, delay release coating include polymers known in the art as enteric coatings for pH-triggered delayed release of pharmaceuticals. Such coatings are impermeable to sertraline at the pH of the stomach, but become permeable in the small intestinal environment, whether by dissolving, disintegrating, or otherwise breaking down, so that sertraline can freely pass through the coating. pH-sensitive polymers which are relatively insoluble and impermeable at the pH of the stomach, but which are more soluble and permeable at the pH of the small intestine and colon include polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, cellulose acetate trimellitate, styrenemaleic acid polyvinylacetate phthalate copolymer, styrene and maleic acid copolymers, polyacrylic acid derivatives such as acrylic acid and acrylic ester copolymers, polymethacrylic acid and esters thereof, poly acrylic methacrylic acid copolymers, shellac, and vinyl acetate and crotonic acid copolymers.

Preferred pH-sensitive polymers include shellac, phthalate derivatives, particularly cellulose acetate phthalate, polyvinylacetate phthalate, and hydroxypropylmethylcellulose phthalate; cellulose acetate trimellitate; polyacrylic acid derivatives, particularly copolymers comprising acrylic acid and at least one acrylic acid ester: polymethyl methacrylate blended with acrylic acid and acrylic ester copolymers; and vinyl acetate and crotonic acid copolymers.

A particularly preferred group of pH-sensitive polymers includes cellulose acetate phthalate, polyvinylacetate phthalate, hydroxypropylmethylcellulose phthalate, cellulose acetate trimellitate, anionic acrylic copolymers of methacrylic acid and methylmethacrylate, and copolymers comprising acrylic acid and at least one acrylic acid ester.

The thickness of the delayed release coating is adjusted to give the desired delay property. In general, thicker coatings are more resistant to erosion and, consequently, yield a longer delay. Preferred coatings range in thickness from about 20 μm to about 1 mm.

When ingested, the release delay means or coating or enteric coating is advantageously adapted so that the unit dosage form or at least the first form passes through the stomach, while preventing the release of SSRI (i.e. maintains a release rate less than 1 mg SSRI/hr) under the acidic conditions prevalent there. After the unit dosage form or the first SSRI containing form passes out of the stomach (wherein certain side effects may be mediated) and into the small intestine, where the pH is comprised between 6.8 and 7.5, the release delay means or coating or enteric coating is eroded, and/or dissolved and/or broken according to the physicochemical properties of the chosen material.

The first form can be multiparticulates or beads wherein each particle or bead is coated with an enteric coating, for example a coating layer with a polymer designed to delay onset of release in the environment of the GI tract when the dosage form is ingested. The multiparticulate or bead may be of a composition and be fabricated by any of the techniques previously disclosed for multiparticulates used to make reservoir systems (including extrusion and spheronization, wet granulation, fluid bed granulation, and rotary bed granulation, seed building, and so forth).

The second form can also be provided with means for preventing the release of the additional active agent within the stomach. For example, the second form can be multiparticles or beads containing the additional active agent, wherein each particle or bead is coated with an enteric coating, for example a coating layer with a polymer designed to delay onset of release in the environment of the GI tract when the dosage form is ingested. The multiparticulate or bead may be of a composition and be fabricated by any of the techniques previously disclosed for multiparticulates used to make reservoir systems (including extrusion and spheronization, wet granulation, fluid bed granulation, and rotary bed granulation, seed building, and so forth).

The first form can also be provided with a sustained release coating as known in the art, so as to control the release rate of SSRI in the intestine.

In general, a lower pH-sensitive polymer content in the coating membrane will give a longer delay. The delay may be further controlled by incorporation, to a lesser or greater degree, of water-soluble polymers such as HPMC, and low molecular weight compounds like glycerol, sucrose, glucose, sodium chloride, citric acid, and fumaric acid. The delay time may be increased by choosing water-soluble membrane porosigens which have lower solubility or slower hydration. For example, citric acid as a membrane coating porosigen, relative to fumaric acid as a membrane coating porosigen, will cause a shorter delay, due to citric acid's higher solubility.

Various delay release means are disclosed in WO 99/01121, the content of which is incorporated by reference. Said document discloses also specific means for ensuring a sustained release for a SSRI.

Other techniques suitable for ensuring delay release are disclosed in US2009/0017113, U.S. Pat. No. 4,693,896, and technical data sheets SURETETIC® and OPADRY® ENTERIC of Colorcon, USA.

In Vitro Dissolution Test: The following in vitro test can be used to screen release embodiments of this invention for in vivo suitability. If a particular dosage form satisfies the in vitro criteria or the in vivo criteria disclosed herein, it is within the scope of this invention.

Release dosage forms of SSRI are tested in a standard USP rotating paddle apparatus as disclosed in United States Pharmacopoeia XXIII (USP) Dissolution Test Chapter 711, Apparatus 2. Paddles are rotated at 50 rpm (or 100 rpm if the dosage form is multiparticulate or disintegrates quickly into multiparticulates) and the dissolution is conducted in, as the test medium, 900 mL acetate buffer (0.13M acetic acid) with 0.075M sodium chloride using potassium hydroxide to adjust pH to 4.0, at 37° C. The dissolution vessels are covered to prevent evaporation. If gelatin capsules are used, then 0.1 mg/mL of the enzyme trypsin must be added to the buffer. At indicated times following test initiation (i.e. insertion of the dosage form into the apparatus), filtered aliquots (typically 2 or 1 0 mL) from the test medium are withdrawn and analyzed for SSRI by reverse-phase high performance liquid chromatography (HPLC) or other suitable quantifiable analysis method. Dissolution results are reported as mg SSRI dissolved versus time or percent of SSRI dissolved versus time.

A convenient test for a spatially delayed release embodiment of the current invention is a modified version of a two part in vitro dissolution test, which is described in the 1995 US. Pharmacopoeia (USP 23), Section [724], Subsection “Delayed Release (Enteric-coated) Articles General Drug Release Standard”, which incorporates a 2 hr test of SSRI release in a simulated gastric fluid (acid test”), followed by a test of drug release in a simulated intestinal fluid (neutral test”). For tablets and capsules which do not contain multiparticulates or disintegrate rapidly into multiparticulates, stirring is effected using paddles at 50 rpm. For multiparticulates or dosage forms that disintegrate into multiparticulates, stirring is effecting using paddles at 100 rpm. If gelatin capsules are used, then 0.1 mg/mL of the enzyme trypsin must be added to the buffer. This two stage in vitro test is adjusted to be useful in evaluating spatially delayed plus sustained embodiments of this invention, as now described.

For pH-triggered spatially-delayed release embodiments, the in vitro test is carried out as described in the USP “Enteric Test”, with the requirements that dosage forms of the invention (a) release sertraline at a rate not exceeding 1 mg SSRI/hr for at least one hour during the acid” phase of the test (in 0.1 N HCI), and (b) release SSRI at a rate between 1 mg SSRI/hr and 40 mg SSRI/hr in the neutral phase of the test. If desired, the acid phase portion of the test can be carried out for longer than 1 hour, i.e., under even more stringent conditions and such embodiments are also within the scope of the invention. The acid portion of the test is carried out in 750 ml 0.1N HCI, for 1 hr. After 1 hr, 250 ml 0.2M tribasic sodium phosphate, containing 10 gram polysorbate-80, is added to the acid medium (containing the dosage form), and the pH is adjusted to pH 6.8, using either 2M hydrochloric acid or 2M sodium hydroxide. The solubility of SSRI is low in phosphate buffer (pH 6.8). Thus polysorbate-80 (1% w/v) is added to the neutral (pH 6.8) phosphate medium to increase the SSRI solubility to provide “sink conditions” for dissolution.

For enzyme-triggered spatially-delayed embodiments described in this disclosure, release of SSRI is “triggered” by the presence of pancreatic lipase, esterase, or protease in the small intestine.

For in vitro evaluation of lipase-triggered delayed plus sustained release dosage forms, 5 mg/ml porcine pancreatic lipase (Sigma Chem., St. Louis, Mo.) is included in the dissolution medium for the second neutral stage of the dissolution test. For esterase- or protease-triggered delayed release systems, appropriate esterase's or protease's (e.g. pancreatic esterase, trypsin, chymotrypsin, elastase) are included in the second stage of the in vitro test. Thus the test is conducted in the same manner as for pH-triggered spatially delayed forms, but the neutral phase is conducted in the presence of an enzyme suitable for triggering the onset of sustained release. If the esterase, protease, or lipase is denatured by polysorbate-80, then the first hour of the “neutral” phase is carried out in the presence of enzyme and absence of polysorbate-80. After one hour in the ‘neutral” phase, 10 g of polysorbate80 is added.

### BRIEF DESCRIPTION OF THE DRAWINGS

FIGS. 1 to 7 are schematic cross section views of pellets, tablets and unit dosage forms of the invention.

### DESCRIPTION OF EXAMPLES

### Example 1

A PROZAC solid form containing 20 mg fluoxetine was coated with a thin water insoluble barrier coating. The so coated solid form was then coated with a water-soluble polymer mixed with ASA (acetyl salicylic acid). The water-soluble outer coating comprised about 5 mg ASA.

### Examples 2 to 5

Example 1 was repeated except that the amount of ASA in the outer coating was respectively 10 mg, 20 mg, 40 mg.

### Examples 6 to 10

Examples 1 to 5 were repeated but with a 10 mg fluoxetine solid form.

### Example 11

A solid form containing 35 mg ASA was prepared.

The form was coated with a (water insoluble) barrier coating. The so coated form was then overcoated with a layer (made of a water-soluble polymer) in which 10 mg fluoxetine is dispersed. An outer protective layer is advantageously added.

### Example 12

Example 11 is repeated except that the fluoxetine containing layer contains 20 mg fluoxetine.

Examples 11 and 12 have been repeated, except that the central ASA solid form contained respectively 10 mg, 20 mg, 40 mg ASA.

It is obvious that the above formulation can be prepared by any techniques, such as spheronisation, so as to prepare microgranules or microspheres of fluoxetine coated with ASA containing layer or microgranules or microspheres of ASA coated with fluoxetine containing layer, with or without intermediate barrier layer.

Examples 1 to 12 have been repeated, except that fluoxetine was replaced by sertraline, paroxetine, citalopram, escitalopram or the s isomer of citalopram, and fluvoxamine.

### Example 13

A citalopram solid form containing 20 mg citalopram was coated with a thin water insoluble barrier coating. The so coated solid form was then coated with a water soluble polymer mixed with ASA. The water-soluble outer coating comprised about 5 mg ASA.

### Examples 14 to 17

Example 13 was repeated except that the amount of ASA in the outer coating was respectively 10 mg, 20 mg, 30 mg and 40 mg.

### Example 18

A solid form containing 35 mg ASA was prepared.

The form was coated with a (water insoluble) barrier coating. The so coated form was then overcoated with a layer (made of a water-soluble polymer) in which 20 mg citalopram is dispersed. An outer protective layer is advantageously added.

### Example 19

Example 18 was repeated except that the solid form contains 25 mg ASA.

### Example 20

A solid form containing 10 mg escitalopram was coated with a thin water insoluble barrier coating. The so coated solid form was then coated with a water-soluble polymer mixed with ASA. The water-soluble outer coating comprised about 5 mg ASA.

### Examples 21 to 24

Example 20 was repeated except that the amount of ASA in the outer coating was respectively 10 mg, 20 mg, 35 mg and 40 mg.

### Example 25

A solid form containing 35 mg ASA was prepared.

The form was coated with a (water insoluble) barrier coating. The so coated form was then overcoated with a layer (made of a water-soluble polymer) in which 10 mg escitalopram is dispersed. An outer protective layer is advantageously added.

### Example 26

Example 25 was repeated except that the solid form contains 25 mg ASA.

### Examples 27 to 38

Examples 1 to 12 have been repeated except that fluoxetine was replaced by Venlafaxin.

### Examples 39 to 74

Examples 1 to 12 have been repeated except that fluoxetine was replaced respectively by Duloxetine, milnacipram and reboxetine.

### Example 75

A capsule (hard gelatin) was filled with first solid forms (such as granules) containing fluoxetine and second solid forms (such as granules) containing ASA. The first solid forms correspond to a dose of 20 mg fluoxetine, while the second solid forms correspond to a dose of 20 mg of ASA. The first solid forms comprise an outer barrier layer for protecting the fluoxetine from the ASA.

### Examples 76 to 80

Example 75 has been repeated, except that fluoxetine was replaced respectively by Citalopram, escitalopram, Duloxetine, milnacipram and reboxetine

### Example 81

A kit is formed with a capsule or tablet containing 20 mg fluoxetine and another capsule or tablet containing 20 mg ASA. The capsules or tablets re advantageously placed in a blister.

### Examples 82 to 86

Example 81 has been repeated except that fluoxetine has been replaced respectively by citalopram, escitalopram, duloxetine, milnacipram and reboxetine.

### Example 83

### Preparation of Enteric Coated Escitalopram Pellets

The pellets were prepared in a fluid bed coating device in the following way.

On beads, such as water soluble beads, for example sugar beads having a size of about 750 μm an escitalopram containing layer was applied. The coating of the beads was carried by using a water dispersion of hydroxypropyl methylcellulose Methocel E5® containing escitalopram, the weight ratio escitalopram/hydroxypropylmethyl cellulose being about 2:1.

After said escitalopram layer was dried, the coated beds were provided with a separating layer. Said separating layer is prepared from an aqueous dispersion of hydroxy propyl methylcellulose Methocel E5® containing polyvinyl pyrolidone (PVP Kollidon K30) and talc.

After said barrier coating being dried, the escitalopram pellets have been provide with an enteric coating layer. The enteric coating layer was achieved by spraying an aqueous dispersion of simethicone, triethylcitrate, talc and an acid resistant methacrylic acid copolymer (such as Eudragit L30 D-55®). The enteric coating layer is dried. Said enteric coating layer was then overcoated with a further enteric coating layer with the same dispersion.

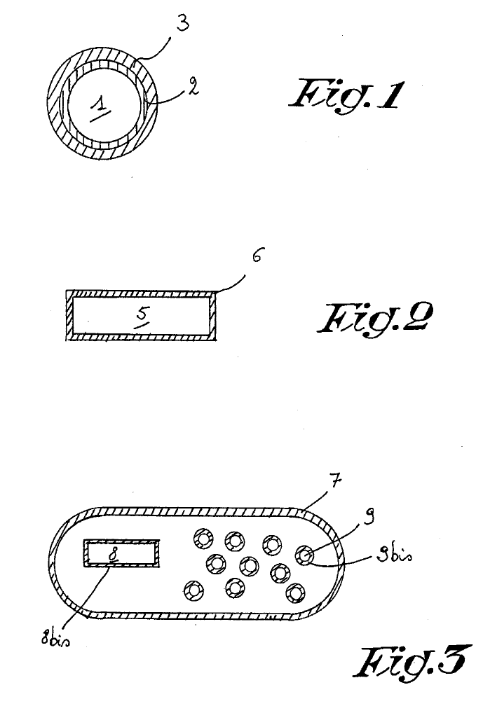

The so prepared coated beads are schematically shown in FIG. 1. In said figure, the following numerals 1, 2, 3 and designate respectively the bead, the escitalopram containing layer, the separating layer, and the enteric coating.

The composition of the pellets, in % by weight, is the following:

| | |
|---|---|
| bead | 20% |
| sugar | 20% |
| escitalopram containing layer | 20% |
| escitalopram | 13% |
| HPMC | 7% |
| Separating layer | 5% |
| HPMC | 2.5% |
| Talc | 1.25% |
| PVP | 1.25% |
| Enteric coating (two layers | 50% |
| Talc | 15.99% |
| Simethicon | 0.01% |
| Triethylcitrate | 4% |
| Eudragit L30 | 30% |

The coated beads were tested for 2 hours in USP apparatus I at 100 rounds per hour, for 2 hours in a medium 0.1N HCl having a pH of 1.2. No escitalopram was released for said two hours testing.

The pellets were further tested for two hours in a simulated gastric fluid described in the 1995 US. Pharmacopoeia (USP 23), Section [724], Subsection “Delayed Release (Enteric-coated) Articles General Drug Release Standard” (see procedure disclosed in WO99/01121). No release of escitalopram was observed in said gastric fluid for said two hours.

Escitalopram coated pellets were subjected to dissolution testing, according to USP 26, Physical Tests/{724} Drug Release, Delayed-release (enteric coated) articles—General drug release standard, Method B, pages 2160 and 2161. The dissolution test is based on USP-711-, “Apparatus 1”. Dissolution in pH 6.8 phosphate buffer USP, performed after 2 hours dissolution in 0.1N HCl. It was observed that the release of escitalopram started after a delay period of more than 10 minutes, namely even more than 20 minutes in the medium at pH 6.8. About 100% by weight of escitalopram were released within two hours following the start of the escitalopram release.

When dissolution tested in vitro in a USP-2 apparatus containing 900 ml of acetate buffer, pH 4.0, which is 0.075 M in NaCl, said USP-2 apparatus being equipped with a paddle stirring at 50 rpm, the escitalopram pellets had a SSRI release at a rate less than 1 mg SSRI/hr for a delay period comprised between 20 minutes and 4 hours.

### Example 84

### Enteric Escitalopram Pellets

Example 83 has been repeated, except that the enteric coating was thinner.

The composition of the pellets, in % by weight, is the following:

| | |
|---|---|
| bead | 50% |
| sugar | 50% |
| escitalopram containing layer | 25% |
| escitalopram | 16% |
| HPMC | 9% |
| Separating layer | 5% |
| HPMC | 2.5% |
| Talc | 1.25% |
| PVP | 1.25% |
| Enteric coating (two layers) | 20% |
| Talc | 6.39% |
| Simethicon | 0.01% |
| Triethylcitrate | 1.6% |
| Eudragit L30 | 12% |

Said pellets prevented release of escitalopram in acid medium, especially in gastric fluid medium. When tested in the pH 6.8 medium, following a 2 hours dissolution in 0.1 N HCl, the release of escitalopram started with a delay of about 10 minutes.

### Example 85

### Enteric Escitalopram Pellets

Example 83 was repeated, except that the enteric coating layer was prepared from a solution of methanol and methylene chloride (about 400 g with a weight ration methanol/methylene chloride of 1:1), said solution containing 30 g of Eudragit RS100, 5 g of Eudragit S10, 5 g of Eudragit L100 and 2 g of macrogol.

Said solution was sprayed on the coated pellets, so that after drying the pellets were provided with an enteric coating representing about 1% of the weight of the enteric coated escitalopram pellets.

No release was observed in the acid medium, while the release at a pH of 7.2 started with a delay of more than 30 minutes.

### Examples 86

Examples 83 to 85 have been repeated, except that the sugar beads have been replaced by glycine betaine beads. The glycine betaine beads were prepared by extrusion/spheronisation.

The use of such beads was interesting as accelerating the disintegration of the pellets, after the delay period.

### Examples 87

Examples 83 to 86 have been repeated, except that the escitalopram has been replaced by another active agent.

Pellets according to the method of preparation of examples 83 to 85 have been prepared so as to comprise respectively as active agent:
fluoxetine,
citalopram,
paroxetine,
venlafaxine,
duloxetine,
dapoxetine,
fluvoxamine,
sertraline.

### Example 88

### Aspirin Tablet Provided with an Enteric Coating

Aspirin tablets (disintegrable when immersed in water) comprising 40 mg ASA were coated with OPADRY® Enteric 940 (Colorcon) mixed in isopropanol and water (with a weight ratio 80 isopropanol: 20 water). The coating dispersion comprised 6% by weight OPADRY®. The aspirin tablets were coated so that the coating, after drying, represents a weight gain of 3% with respect to the tablet before coating.

Substantially no aspirin was released from the coated tablets at pH 1.2, as well as at pH 5.5. Disintegration of the tablets started at pH 6.8, and was very rapid (less than 10 minutes).

Such a coated aspirin tablet is schematically represented in FIG. 2, in which numeral 5 designates the core of the tablet, while numerals 6 designates the surrounding enteric coating.

### Example 89

### Aspirin Tablet Provided with an Enteric Coating

Aspirin tablets (disintegrable in water) comprising 40 mg ASA have been provided with an ethylcellulose coating. The tablets were coated by using the method disclosed in U.S. Pat. No. 4,693,896, using the coating formulation 1. The so coated tablets, after drying, had a thin coating representing about 0.7% of weight of the coated tablet.

Such tablets are rapidly disintegrated in the intestine, while passing intact through the stomach.

### Examples 90

### Of Unit Dosage Forms Using Water Soluble Capsule

Soft gelatin capsules have been used. The size of which was adapted for containing the coated aspirin tablet, as well as enteric coated SSRI pellets.

Such a soft gelatin capsule is soluble or disintegrable within the stomach, whereby liberating the enteric coated SSRI pellets and the enteric coated aspirin tablet.

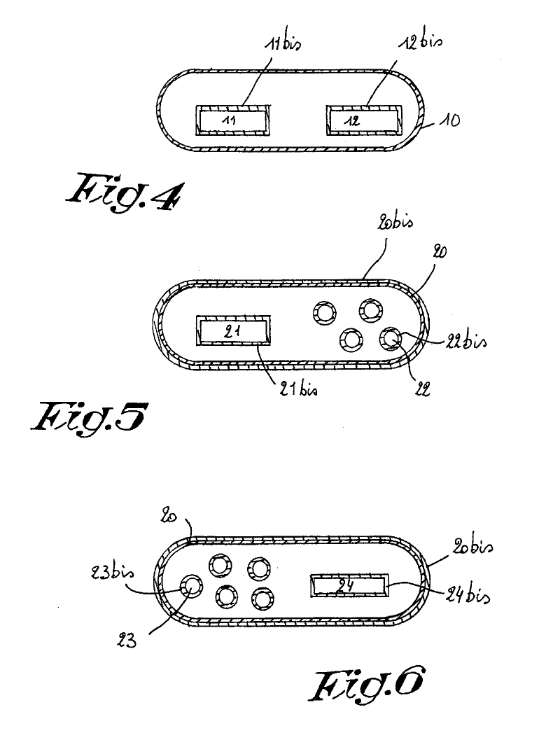

FIG. 3 is a schematic view in cross section of such a capsule, in which numerals 7 designates the shell of the capsule, while numerals 8 and 9 designate respectively the SSRI pellets and the aspirin tablet.

### Example 90

### Aspirin Tablet Provided with an Enteric Coating

Aspirin tablets (disintegrable when immersed in water) comprising 40 mg ASA and 100 mg glycine betaine (with a binder) were prepared and then coated with OPADRY® Enteric 940 (Colorcon) mixed in isopropanol and water (with a weight ratio 80 isopropanol: 20 water). The coating dispersion comprised 6% by weight OPADRY®. The aspirin tablets were coated so that the coating, after drying, represents a weight gain of 3% with respect to the tablet before coating.

Substantially no aspirin was released from the coated tablets at pH 1.2, as well as at pH 5.5. Disintegration of the tablets started at pH 6.8, and was very rapid (less than 10 minutes).

Such a coated aspirin tablet is schematically represented in FIG. 2, in which numeral 5 designates the core of the tablet, while numerals 6 designates the surrounding enteric coating.

### Example 91

### Aspirin Tablet Provided with an Enteric Coating

Aspirin tablets (disintegrable in water) comprising 40 mg ASA and 200 mg glycine betaine have been prepared with a binder (such as Emcompress® or Emdex® from JRS Pharma, USA) and then provided with an ethylcellulose coating. The tablets were coated by using the method disclosed in U.S. Pat. No. 4,693,896, using the coating formulation 1. The so coated tablets, after drying, had a thin coating representing about 0.7% of weight of the coated tablet.

Such tablets are rapidly disintegrated in the intestine, while passing intact through the stomach.

### Examples 92

### Of Unit Dosage Forms Using Water Soluble Capsule

Soft gelatin capsules have been used. The size of which was adapted for containing the coated aspirin tablet, as well as enteric-coated SSRI pellets.

Such a soft gelatin capsule is soluble or disintegrable within the stomach, whereby liberating the enteric coated SSRI pellets and the enteric coated aspirin tablet.

A unit dosage form is schematically represented in FIG. 3, in which numerals 7, 8 and 9 designate respectively the shell of the capsule, the ASA tablet and the pellets. The enteric coatings are designed by 8bis and 9bis.

The following capsules were prepared:

| | ASA | | | |
|---|---|---|---|---|
| | tablet | | | |
| | example | | dose | Pellets |
| | 88 dose | | SSRI | prepared by the method of |
| capsule | (mg) | SSRI | mg | example |
| 1 | 40 | escitalopram | 20 | 83 |
| 2 | 40 | escitalopram | 15 | 83 |
| 3 | 40 | escitalopram | 10 | 83 |
| 4 | 40 | escitalopram | 20 | 84 |
| 5 | 40 | escitalopram | 15 | 84 |
| 6 | 40 | escitalopram | 10 | 84 |
| 7 | 40 | escitalopram | 20 | 85 |
| 8 | 40 | escitalopram | 15 | 85 |
| 9 | 40 | escitalopram | 10 | 85 |
| 10 | 40 | escitalopram | 20 | 86 (83 + glycine betaine beads) |
| 11 | 40 | escitalopram | 20 | 86 (84 + glycine betaine beads) |
| 12 | 40 | escitalopram | 20 | 86 (85 + glycine betaine beads) |
| 13 | 40 | citalopram | 20 | 83 |
| 14 | 40 | citalopram | 15 | 83 |
| 15 | 40 | citalopram | 10 | 83 |
| 16 | 40 | citalopram | 20 | 84 |
| 17 | 40 | citalopram | 15 | 84 |
| 18 | 40 | citalopram | 10 | 84 |
| 19 | 40 | citalopram | 20 | 85 |
| 20 | 40 | citalopram | 15 | 85 |
| 21 | 40 | citalopram | 10 | 85 |
| 22 | 40 | citalopram | 20 | 86 (83 + glycine betaine beads) |
| 23 | 40 | citalopram | 20 | 86 (84 + glycine betaine beads) |
| 24 | 40 | citalopram | 20 | 86 (85 + glycine betaine beads) |
| 25 | 40 | fluoxetine | 20 | 83 |
| 26 | 40 | fluoxetine | 15 | 83 |
| 27 | 40 | fluoxetine | 10 | 83 |
| 28 | 40 | fluoxetine | 20 | 84 |
| 29 | 40 | fluoxetine | 15 | 84 |
| 30 | 40 | fluoxetine | 10 | 84 |
| 31 | 40 | fluoxetine | 20 | 85 |
| 32 | 40 | fluoxetine | 15 | 85 |
| 33 | 40 | fluoxetine | 10 | 85 |
| 34 | 40 | fluoxetine | 20 | 86 (83 + glycine betaine beads) |
| 35 | 40 | fluoxetine | 20 | 86 (84 + glycine betaine beads) |
| 36 | 40 | fluoxetine | 20 | 86 (85 + glycine betaine beads) |
| 37 | 40 | paroxetine | 20 | 83 |
| 38 | 40 | paroxetine | 15 | 83 |
| 39 | 40 | paroxetine | 10 | 83 |
| 40 | 40 | paroxetine | 20 | 84 |
| 41 | 40 | paroxetine | 15 | 84 |
| 42 | 40 | paroxetine | 10 | 84 |
| 43 | 40 | paroxetine | 20 | 85 |
| 44 | 40 | paroxetine | 15 | 85 |
| 45 | 40 | paroxetine | 10 | 85 |
| 46 | 40 | paroxetine | 20 | 86 (83 + glycine betaine beads) |
| 47 | 40 | paroxetine | 20 | 86 (84 + glycine betaine beads) |
| 48 | 40 | paroxetine | 20 | 86 (85 + glycine betaine beads) |
| 49 | 40 | venlafaxine | 40 | 83 |
| 50 | 40 | venlafaxine | 20 | 83 |
| 51 | 40 | duloxetine | 20 | 83 |
| 52 | 40 | venlafaxine | 40 | 84 |
| 53 | 40 | venlafaxine | 20 | 84 |
| 54 | 40 | duloxetine | 20 | 84 |
| 55 | 40 | venlafaxine | 40 | 85 |
| 56 | 40 | venlafaxine | 20 | 85 |
| 57 | 40 | duloxetine | 20 | 85 |
| 58 | 40 | venlafaxine | 20 | 86 (83 + glycine betaine beads) |
| 59 | 40 | venlafaxine | 20 | 86 (84 + glycine betaine beads) |
| 60 | 40 | duloxetine | 20 | 86 (85 + glycine betaine beads) |
| 61 | 40 | dapoxetine | 20 | 83 |
| 62 | 40 | fluvoxamine | 20 | 83 |
| 63 | 40 | sertraline | 20 | 83 |
| 64 | 40 | dapoxetine | 20 | 84 |
| 65 | 40 | fluvoxamine | 20 | 84 |
| 66 | 40 | sertraline | 20 | 84 |
| 67 | 40 | dapoxetine | 20 | 85 |
| 68 | 40 | fluvoxamine | 20 | 85 |
| 69 | 40 | sertraline | 20 | 85 |
| 70 | 40 | dapoxetine | 20 | 86 (83 + glycine betaine beads) |
| 71 | 40 | fluvoxamine | 20 | 86 (84 + glycine betaine beads) |
| 72 | 40 | sertraline | 20 | 86 (85 + glycine betaine beads) |
| 73 | 40 | dapoxetine | 40 | 83 |
| 74 | 40 | fluvoxamine | 40 | 83 |
| 76 | 40 | sertraline | 40 | 83 |
| 77 | 40 | dapoxetine | 40 | 84 |
| 78 | 40 | fluvoxamine | 40 | 84 |
| 79 | 40 | sertraline | 40 | 84 |
| 80 | 40 | dapoxetine | 40 | 85 |
| 81 | 40 | fluvoxamine | 40 | 85 |
| 82 | 40 | sertraline | 40 | 85 |
| 83 | 40 | dapoxetine | 40 | 86 (83 + glycine betaine beads) |
| 84 | 40 | fluvoxamine | 40 | 86 (84 + glycine betaine beads) |
| 85 | 40 | sertraline | 40 | 86 (85 + glycine betaine beads) |

### Example 91

### SSRI Sustained Release Hydrophilic Matrix Tablets

Tablets were prepared by blending the following components: SSRI 30% by weight, 25% by weight Methocel K100 LV (Dow Chemical), 5% by weight Methocel K4M (Dow Chemical), 40% by weight Emcompress (JRS), and 1% by weight magnesium stearate, and then by compressing the blend into tablets.

The tablets were then coated with an enteric coating comprising by weight:

| | |
|---|---|
| Talc | 27.9% |
| Simethicon | 0.1% |
| Triethylcitrate | 9% |
| Eudragit L30 | 63% |

Tablets were provided with an enteric coating representing about 20% of the weight of the uncoated sustained tablet. Possibly, tablets can be provided with two enteric coating layers representing each about 20% of the weight of the uncoated sustained tablet.

When using sustained tablets, dosage form can comprise higher amount of SSRI.

### Example 92

### Capsules Comprising Enteric Coated Sustained Release SSRI Tablet+Enteric Coated ASA Tablet

A soft gelatin capsule is soluble or disintegrable within the stomach, whereby liberating the enteric coated SSRI tablet and the enteric coated aspirin tablet.

A unit dosage form is schematically represented in FIG. 4, in which numerals 10, 11 and 12 designate respectively the shell of the capsule, the ASA tablet and the SSRI tablet. 11bis and 12bis designate the enteric coatings.

Capsules comprising an enteric-coated ASA tablet (40 mg)

| | SSRI in | SSRI dose |
|---|---|---|
| Capsule | the tablet | (mg) |
| 1 | escitalopram | 20 |
| 2 | escitalopram | 40 |
| 3 | citalopram | 20 |
| 4 | citalopram | 40 |
| 5 | paroxetine | 20 |
| 6 | paroxetine | 40 |
| 7 | venlafaxine | 80 |
| 8 | venlafaxine | 60 |
| 9 | venlafaxine | 40 |
| 10 | venlafaxine | 20 |
| 11 | duloxetine | 40 |
| 12 | duloxetine | 20 |
| 13 | dapoxetine | 80 |
| 14 | dapoxetine | 60 |
| 15 | dapoxetine | 40 |
| 16 | dapoxetine | 20 |
| 17 | fluvoxamine | 80 |
| 18 | fluvoxamine | 40 |
| 19 | fluvoxamine | 20 |
| 20 | sertraline | 80 |
| 21 | setraline | 40 |
| 22 | sertraline | 20 |

### Examples 93

### Capsule Provided with an Outer Surrounding Enteric Coating

Hard gelatin capsules are provided with an outer enteric coating, said coating being adapted for preventing any drug release within the stomach.

The coating of the capsule was for example:

| | |
|---|---|
| Talc | 27.9% |
| Simethicon | 0.1% |
| Triethylcitrate | 9% |
| Eudragit L30 | 63% |

The coating, after drying, represented about 30% of the weight of the coated capsule comprising the SSRI and the ASA.

The capsule was first filled with SSRI pellets or tablet provided with an enteric coating adapted for ensuring some delay of release, and with ASA pellets or tablet ensuring an immediate release into the intestine, as soon as the capsule is broken.

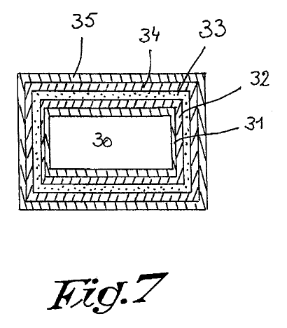

FIGS. 5 and 6 show schematically respectively:
a enteric coated capsule 20 comprising an enteric coated SSRI containing tablet 21 and ASA pellets 22, the enteric coating being designated by 20bis, 21 bis and 22bis, and an enteric coated capsule 20 comprising enteric coated SSRI containing pellets 23 and an ASA tablet 24 provided with a barrier layer 25 (water soluble), the enteric coatings being designated by 20bis, 23bis and 24bis.

The aspirin Tablet can be prepared by blending ASA, optionally glycine betaine, and a binder (such as Emcompress® or Emdex® from JRS Pharma, USA. Optionally one or more further excipient can be added.

After tabletting, the ASA tablet can be provided with a thin protecting layer, made of a water-soluble polymer or copolymer.

The aspirin pellets can be prepared by spray drying on beads, such as sugar beads or glycine betaine beads an ASA dispersion, and then by overcoating said ASA layer with a barrier layer.

The composition of the ASA pellets, in % by weight, is for example the following:

| | |
|---|---|
| bead | 60% |
| sugar and/or glycine betaine | 60% |
| ASA containing layer | 30% |
| ASA | 20% |
| HPMC | 10% |
| barrier layer | 10% |
| HPMC | 5% |
| Talc | 2.5% |
| PVP | 2.5% |

The SSRI pellets or tablets provided with an enteric coating layer is for example prepared by one the method disclosed in examples 83 to 87.

### Example 94

### SSRI Tablets Provided with a Surrounding ASA Layer and with an Enteric Outer Layer

Such a tablet is schematically shown in FIG. 7.

SSRI tablet (30) can be prepared by blending SSRI with tabletting agents (such as Emcompress®-JRS) and by compressing the blending. The tablet can advantageously be a sustained release tablet, such as disclosed in example 91.

The SSRI tablet (30) is provided with a enteric coating (31), for example a coating comprising:

| | |
|---|---|
| Talc | 27.9% |
| Simethicon | 0.1% |
| Triethylcitrate | 9% |
| Eudragit L30 | 63% |

The coating represent for example 10 to 20% by weight of the SSRI tablet.

A barrier layer (32) surrounds the enteric layer, the weight of said layer being for example comprised between 50 and 100% of the weight of the enteric coating. The barrier layer is water soluble or disintegrable. The composition of the barrier layer is:

| | |
|---|---|
| HPMC | 50% |
| Talc | 25% |
| PVP | 25% |

An ASA containing coating (33) is then applied on said barrier layer. Said ASA containing layer comprises 40 mg ASA, and 20 mg hydroxypropyl methyl cellulose.

A barrier layer (34) surrounds the ASA layer; the weight of said layer being for example comprised between 50 and 100% of the weight of the ASA coating. The barrier layer is water soluble or disintegrable. The composition of the barrier layer is:

| | |
|---|---|
| HPMC | 50% |
| Talc | 25% |
| PVP | 25% |

An outer enteric layer (35) surrounds the latter barrier layer, said enteric layer being for example:

| | |
|---|---|
| Talc | 27.9% |
| Simethicon | 0.1% |
| Triethylcitrate | 9% |
| Eudragit L30 | 63% |

The coating represent for example 10 to 20% by weight of the SSRI tablet provided with its entire coating layer.

It is obvious that said examples can be carried out with other antidepressant active agents, and possibly, if required with larger ASA dose, especially when the ASA containing form is a controlled release form, for example releasing ASA at specific moments with intermediate period without ASA release.

Instead of using ASA, salt and esters thereof as well as diaspirin can be used.

Tests Made on Rats

Four groups of rats have been stressed. One group of rats remain unstressed so as to serve as control.

After being stressed, the rats of the first group received a dose of 50 mg ASA per kg. The second group of rats received no treatment. The third group of rats received 5 mg fluoxetine per kg, and the fourth group of rats received simultaneously 5 mg fluoxetine+50 mg ASA per kg.

The treatment was continued during 3 weeks.

The control group of rats had an activity level of 30, while the stressed rats had an activity of 2.

The result of this animal test is:
after 1 week of treatment with the combination fluoxetine+ASA, the fourth group of rats had an activity of 30, i.e. corresponding to the activity of the unstressed rats or control rats. After 1 week of treatment with ASA or fluoxetine alone, there was no increase at all of the activity.
after three weeks of treatment, the fourth group of rats had still an activity of 30. The third group of rats (treated with fluoxetine) had after 3 weeks an activity of about 25, while no improvement of activity was observed with ASA.

In said test, the doses of ASA and fluoxetine have been adapted for rats.

Said test shows clearly the unexpected action or synergistic activity of ASA+fluoxetine. While not being bound to any theory, it is believed that ASA promotes or activates the sites on which the fluoxetine has to bind.

Further tests have been carried out with lower doses of ASA, namely 25 mg. Similar onset time of antidepressant action was observed.

In view of the reducing time of onset of antidepressant action obtained for the family of SSRI antidepressant, it can be concluded that ASA at dose lower than 50 mg is also efficient for reducing the time of antidepressant onset action for pharmaceutically acceptable antidepressant active agents selected from the group consisting of SNRIs (serotonin noradrenaline reuptake inhibitors), CRF (corticotropic releasing factor) antagonists, NK1 (neurokinin) antagonists, NK2 (neurokinin) antagonists, NK3 (neurokinin) antagonists and combinations thereof, possibly mixed with one or more SSRI.

From said tests and informal tests made on volunteers, in which after (just after, less than 5 minutes after) and prior oral administration of an antidepressant, a dose of ASA was orally administered, it appears that the oral dose of ASA had to be lowered to less than 50 mg, especially between 5 and 50 mg.

At such a low dose of ASA, no bleeding was observed, but a very quick onset of antidepressant action was observed. From this test, it seems that the antidepressant action could already be achieved in less than 7 days, and even in less than 3 to 4 days, although when not using low dose of ASA, the antidepressant action is for example of at least 4 weeks for SSRI.

A comparative test was made on rats by combining a COX II inhibitor with a SSRI (fluoxetine). After 1 week treatment, an antidepressant action could be seen, the efficiency of said combination being however less than 50% of the efficiency of the combination ASA+fluoxetine.

Higher dose of ASA (dose of 100 mg for example) administered to SSRI responding human seems to have no influence or even to have a negative influence on the time of onset of antidepressant action on some SSRI responding patients.

Tests made on SSRI resistant patients or SSRI not responding patients have shown that the combination of the invention, possibly with higher dose of ASA, was suitable for rendering more than 50% of said patients responding quickly to SSRI.

What we claim is:

1. An oral antidepressant formulation for human comprising an effective antidepressant amount of at least one pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof, and an effective amount lower than 50 mg of at least one compound selected from the group consisting of acetylsalicylic acid, salts and esters of acetylsalicylic acid, diaspirin, and mixtures thereof, for reducing the onset of antidepressant action of the pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof.

2. The oral formulation of claim 1, which comprises a means for controlling the release of the pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof, with respect to the release of the compound selected from the group consisting of acetylsalicylic acid, salts and esters of acetylsalicylic acid, diaspirin, and mixtures thereof.

3. The oral formulation of claim 1, which comprises a means for delaying the start of the release of the pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof, from 3 minutes to 60 minutes with respect to the release of at least 50% by weight of the effective amount of the compound selected from the group consisting of acetylsalicylic acid, salts and esters of acetylsalicylic acid, diaspirin, and mixtures thereof.

4. The oral formulation of claim 1, which comprises an effective amount of at least a compound selected from the group consisting of acetylsalicylic acid, salts and esters of acetylsalicylic acid, diaspirin, and mixtures thereof, for reducing at least 15% the average onset of action of the pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof.

5. The formulation of claim 1, in which the effective amount of the compound for reducing the onset of action of the pharmaceutically acceptable antidepressant active agent selected of the group consisting of SSRI agents, SNRIs (serotonin noradrenaline reuptake inhibitors), CRF antagonists, NK1 antagonists, NK2 antagonists, NK3 antagonists and combinations thereof is lower than 5 times the amount of pharmaceutically acceptable antidepressant active agents preferably selected from the group consisting of SSRI agents, SNRIs (serotonin noradrenaline reuptake inhibitors), CRF antagonists, NK1 antagonists, NK2 antagonists, NK3 antagonists and combinations thereof.

6. The formulation of claim 1, which comprises a means for delaying the release of the pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof, from 5 minutes to 45 minutes with respect to the release of at least 50% by weight of the effective amount of the compound selected from the group consisting of acetylsalicylic acid, salts and esters of acetylsalicylic acid, diaspirin, and mixtures thereof.

7. The formulation of claim 1, which comprises a means for delaying the start of the release of the pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof, from 7 minutes to 30 minutes with respect to the release of at least 50% by weight of the effective amount of the compound selected from the group consisting of acetylsalicylic acid, esters and salts of acetylsalicylic acid, diaspirin, and mixtures thereof.

8. The formulation of claim 1, which comprises a form containing the pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof, said form being provided with a coating for delaying the release of the antidepressant agent with respect to the release of the compound selected from the group consisting of acetylsalicylic acid, esters and salts of acetylsalicylic acid, diaspirin, and mixtures thereof.

9. The formulation of claim 1, which comprises a form containing the pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof, said form being provided with a coating for delaying from 3 minutes to 60 minutes the start of the release of the antidepressant agent with respect to the release of at least 50% by weight of the effective amount of the compound selected from the group consisting of acetylsalicylic acid, esters and salts of acetylsalicylic acid, diaspirin, and mixtures thereof.

10. The formulation of claim 1, which comprises a form containing the pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof, said form being provided with a coating for delaying from 5 minutes to 45 minutes the start of the release of the antidepressant agent with respect to the release of at least 50% by weight of the effective amount of the compound selected from the group consisting of acetylsalicylic acid, esters and salts of acetylsalicylic acid, diaspirin, and mixtures thereof.

11. The formulation of claim 1, which comprises a form containing the pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof, said form being provided with a coating for delaying from 7 minutes to 30 minutes the start of the release of the antidepressant agent with respect to the release of at least 50% by weight of the effective amount of the compound selected from the group consisting of acetylsalicylic acid, esters and salts of acetylsalicylic acid, diaspirin, and mixtures thereof.

12. The formulation of claim 1, which comprises a form selected from the group consisting of particles, core, microparticles, beads and combinations thereof, whereby said form selected from the group consisting of particles, core, microparticles, beads and combinations thereof is provided with a coating comprising at least a compound selected from the group consisting of acetylsalicylic acid, esters and salts of acetylsalicylic acid, diaspirin and mixtures thereof, for reducing the onset of action of the pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof, said coating being substantially free of selective pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof.

13. The formulation of claim 1, which is a solid form comprising from 4 mg up to 100 mg pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof.

14. The formulation of claim 1, which is a solid form comprising from 4 mg up to 20 mg pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof.

15. The formulation of claim 1, which comprises from 5 to 40 mg acetylsalicylic acid, esters and salts of acetylsalicylic acid, diaspirin, and mixtures thereof.

16. The formulation of claim 1, which comprises from 5 to 35 mg acetylsalicylic acid, esters and salts of acetylsalicylic acid, diaspirin, and mixtures thereof.

17. An oral antidepressant formulation for human comprising an effective antidepressant amount of at least one pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof, and an effective amount of at least one compound selected from the group consisting of acetylsalicylic acid, esters and salts of acetylsalicylic acid, diaspirin, and mixtures thereof, for reducing the onset of action of the pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof, whereby said formulation comprises a means for controlling the release of the pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof, with respect to the release of the compound selected from the group consisting of acetylsalicylic acid, esters and salts of acetylsalicylic acid, diaspirin, and mixtures thereof.

18. The formulation of claim 17, which comprises a means for delaying the start of the release of the pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof, from 3 minutes to 60 minutes with respect to the release of at least 50% by weight of the effective amount of the compound selected from the group consisting of acetylsalicylic acid, esters and salts of acetylsalicylic acid, diaspirin, and mixtures thereof.

19. The formulation of claim 18, which comprises a means for delaying the start of the release of the pharmaceutically acceptable antidepressant active agent selected from the group consisting of SSRI agents, SNRIs (serotonin noradrenaline reuptake inhibitors), CRF antagonists, NK1 antagonists, NK2 antagonists, NK3 antagonists and combinations thereof, from 5 minutes to 45 minutes with respect to the release of at least 50% by weight of the effective amount of the compound selected from the group consisting of acetylsalicylic acid, esters and salts of acetylsalicylic acid, diaspirin, and mixtures thereof.

20. A unit dosage form suitable for oral administration to a human comprising:
a first SSRI containing form comprising an effective antidepressant amount of a SSRI compound selected from the group consisting of fluoxetine, citalopram, escitalopram, paroxetine, venlafaxine, duloxetine, dapoxetine, fluvoxamine, sertraline, and pharmaceutically acceptable salts thereof, at least one pharmaceutically acceptable carrier,
at least one release delay means for the SSRI compound in an amount adapted for ensuring at least a delay release period selected from the group consisting of a delay release period at a pH between 1 and 4.5 of at least 20 minutes with a rate of release of less than 2 mg SSRI per hour, a delay release period being the sum of a first delay release period at pH comprised between 1 and 4.5 of at least 20 minutes with a rate of release of less than 2 mg SSRI per hour and of a second delay release period at pH comprised between 6.8 and 7.5 of at least 10 minutes with a rate of release of less than 2 mg SSRI per hour, and
a second form comprising an effective amount of less than 50 mg of an additional active agent selected from the group consisting of acetyl salicylic acid, pharmaceutically acceptable salts thereof, pharmaceutically acceptable esters thereof, diaspirin and mixtures thereof,
whereby said unit dosage form is adapted so that at least 90% by weight of said additional active agent of the second form is released within the said delay release period for the SSRI compound selected from the group consisting of a delay release period at a pH between 1 and 4.5 of at least 20 minutes with a rate of release of less than 2 mg SSRI per hour, a delay release period being the sum of a first delay release period at pH comprised between 1 and 4.5 of at least 20 minutes with a rate of release of less than 2 mg SSRI per hour and of a second delay release period at pH comprised between 6.8 and 7.5 of at least 10 minutes with a rate of release of less than 2 mg SSRI per hour.

21. The unit dosage form of claim 20 comprising:
a first SSRI containing form comprising an effective antidepressant amount of a SSRI compound selected from the group consisting of fluoxetine, citalopram, escitalopram, paroxetine, venlafaxine, duloxetine, dapoxetine, fluvoxamine, sertraline, and pharmaceutically acceptable salts thereof, at least one pharmaceutically acceptable carrier,
at least one release delay means for the SSRI compound in an amount adapted for ensuring at least a delay release period selected from the group consisting of a delay release period at a pH between 1 and 4.5 of at least 60 minutes with a rate of release of less than 2 mg SSRI per hour, a delay release period being the sum of a first delay release period at pH comprised between 1 and 4.5 of at least 60 minutes with a rate of release of less than 2 mg SSRI per hour and of a second delay release period at pH comprised between 6.8 and 7.5 of at least 10 minutes with a rate of release of less than 2 mg SSRI per hour, and
a second form comprising an effective amount of less than 50 mg of an additional active agent selected from the group consisting of acetyl salicylic acid, pharmaceutically acceptable salts thereof, pharmaceutically acceptable esters thereof, diaspirin and mixtures thereof,
whereby said unit dosage form is adapted so that at least 90% by weight of said additional active agent of the second form is released within the said delay release period for the SSRI compound selected from the group consisting of a delay release period at a pH between 1 and 4.5 of at least 60 minutes with a rate of release of less than 2 mg SSRI per hour, a delay release period being the sum of a first delay release period at pH comprised between 1 and 4.5 of at least 60 minutes with a rate of release of less than 2 mg SSRI per hour and of a second delay release period at pH comprised between 6.8 and 7.5 of at least 10 minutes with a rate of release of less than 2 mg SSRI per hour.

22. The unit dosage form of claim 20 comprising:
a first SSRI containing form comprising an effective antidepressant amount of a SSRI compound selected from the group consisting of fluoxetine, citalopram, escitalopram, paroxetine, venlafaxine, duloxetine, dapoxetine, fluvoxamine, sertraline, and pharmaceutically acceptable salts thereof, at least one pharmaceutically acceptable carrier,
at least one release delay means for the SSRI compound in an amount adapted for ensuring at least a delay release period selected from the group consisting of a delay release period at a pH between 1 and 4.5 of at least 60 minutes with a rate of release of less than 2 mg SSRI per hour, a delay release period being the sum of a first delay release period at pH comprised between 1 and 4.5 of at least 60 minutes with a rate of release of less than 2 mg SSRI per hour and of a second delay release period at pH comprised between 6.8 and 7.5 of at least 20 minutes with a rate of release of less than 2 mg SSRI per hour, and
a second form comprising an effective amount of less than 50 mg of an additional active agent selected from the group consisting of acetyl salicylic acid, pharmaceutically acceptable salts thereof, pharmaceutically acceptable esters thereof, diaspirin and mixtures thereof,
whereby said unit dosage form is adapted so that at least 90% by weight of said additional active agent of the second form is released within the said delay release period for the SSRI compound selected from the group consisting of a delay release period at a pH between 1 and 4.5 of at least 60 minutes with a rate of release of less than 2 mg SSRI per hour, a delay release period being the sum of a first delay release period at pH comprised between 1 and 4.5 of at least 60 minutes with a rate of release of less than 2 mg SSRI per hour and of a second delay release period at pH comprised between 6.8 and 7.5 of at least 20 minutes with a rate of release of less than 2 mg SSRI per hour.

23. The unit dosage form of claim 20 comprising:
a first SSRI containing form comprising an effective antidepressant amount of a SSRI compound selected from the group consisting of fluoxetine, citalopram, escitalopram, paroxetine, venlafaxine, duloxetine, dapoxetine, fluvoxamine, sertraline, and pharmaceutically acceptable salts thereof, at least one pharmaceutically acceptable carrier,
at least one release delay means for the SSRI compound in an amount adapted for ensuring at least a delay release period selected from the group consisting of a delay release period at a pH between 1 and 4.5 of at least 120 minutes with a rate of release of less than 2 mg SSRI per hour, a delay release period being the sum of a first delay release period at pH comprised between 1 and 4.5 of at least 120 minutes with a rate of release of less than 2 mg SSRI per hour and of a second delay release period at pH comprised between 6.8 and 7.5 of at least 20 minutes with a rate of release of less than 2 mg SSRI per hour, and
a second form comprising an effective amount of less than 50 mg of an additional active agent selected from the group consisting of acetyl salicylic acid, pharmaceutically acceptable salts thereof, pharmaceutically acceptable esters thereof, diaspirin and mixtures thereof,
whereby said unit dosage form is adapted so that at least 90% by weight of said additional active agent of the second form is released within the said delay release period for the SSRI compound selected from the group consisting of a delay release period at a pH between 1 and 4.5 of at least 120 minutes with a rate of release of less than 2 mg SSRI per hour, a delay release period being the sum of a first delay release period at pH comprised between 1 and 4.5 of at least 120 minutes with a rate of release of less than 2 mg SSRI per hour and of a second delay release period at pH comprised between 6.8 and 7.5 of at least 20 minutes with a rate of release of less than 2 mg SSRI per hour.

24. The unit dosage form of claim 20, comprising:
a first SSRI containing form comprising an effective antidepressant amount of a SSRI compound selected from the group consisting of fluoxetine, citalopram, escitalopram, paroxetine, venlafaxine, duloxetine, dapoxetine, fluvoxamine, sertraline, and pharmaceutically acceptable salts thereof, at least one pharmaceutically acceptable carrier, and at least one release delay means in an amount adapted for ensuring at least a delay release period selected from the group consisting of a delay release period at a pH between 1 and 4.5 of at least 60 minutes with a rate of release of less than 2 mg SSRI per hour, a delay release period being the sum of a first delay release period at pH comprised between 1 and 4.5 of at least 60 minutes with a rate of release of less than 2 mg SSRI per hour and of a second delay release period at pH comprised between 6.8 and 7.5 of at least 10 minutes with a rate of release of less than 2 mg SSRI per hour, and
a second form comprising an effective amount of less than 50 mg of an additional active agent selected from the group consisting of acetyl salicylic acid, pharmaceutically acceptable salts thereof, pharmaceutically acceptable esters thereof, diaspirin and mixtures thereof, whereby said form is adapted so that at least 90% by weight of said additional active agent is released within the said delay release period for the SSRI compound selected from the group consisting of a delay release period at a pH between 1 and 4.5 of at least 60 minutes with a rate of release of less than 2 mg SSRI per hour, a delay release period being the sum of a first delay release period at pH comprised between 1 and 4.5 of at least 60 minutes with a rate of release of less than 2 mg SSRI per hour and of a second delay release period at pH comprised between 6.8 and 7.5 of at least 10 minutes with a rate of release of less than 2 mg SSRI per hour.

25. The unit dosage form of claim 20, comprising:
a first SSRI containing form comprising an effective antidepressant amount of a SSRI compound selected from the group consisting of fluoxetine, citalopram, escitalopram, paroxetine, venlafaxine, duloxetine, dapoxetine, fluvoxamine, sertraline, and pharmaceutically acceptable salts thereof, at least one pharmaceutically acceptable carrier, and at least one release delay means in an amount adapted for ensuring at least a delay release period selected from the group consisting of a delay release period at a pH between 1 and 4.5 of at least 120 minutes with a rate of release of less than 2 mg SSRI per hour, a delay release period being the sum of a first delay release period at pH comprised between 1 and 4.5 of at least 120 minutes with a rate of release of less than 2 mg SSRI per hour and of a second delay release period at pH comprised between 6.8 and 7.5 of at least 10 minutes with a rate of release of less than 2 mg SSRI per hour, and
a second form comprising an effective amount of less than 50 mg of an additional active agent selected from the group consisting of acetyl salicylic acid, pharmaceutically acceptable salts thereof, pharmaceutically acceptable esters thereof, diaspirin and mixtures thereof, whereby said form is adapted so that at least 90% by weight of said additional active agent is released within the said delay release period for the SSRI compound selected from the group consisting of a delay release period at a pH between 1 and 4.5 of at least 120 minutes with a rate of release of less than 2 mg SSRI per hour, a delay release period being the sum of a first delay release period at pH comprised between 1 and 4.5 of at least 120 minutes with a rate of release of less than 2 mg SSRI per hour and of a second delay release period at pH comprised between 6.8 and 7.5 of at least 10 minutes with a rate of release of less than 2 mg SSRI per hour.

26. The unit dosage form of claim 20, which comprises an envelope containing at least the first SSRI containing form and the second form containing the additional active agent, whereby the envelope comprises at least one enteric coating, said envelope being adapted for preventing substantially any release of the SSRI compound and the additional active agent in the stomach, said envelope being selected from the group consisting of envelope rendered permeable at pH comprised between 6 and 7.5, envelope disintegrable at pH comprised between 6 and 7.5, envelope solubilized at pH comprised between 6 and 7.5, and combinations thereof and whereby the first SSRI containing form is provided with a delay release means for ensuring a delay release period of at least 10 minutes for the SSRI compound at pH comprised between 6.8 and 7.5 with a rate of release of less than 2 mg SSRI per hour.

27. The unit dosage form of claim 20, which comprises an envelope containing at least the first SSRI containing form and the second form containing the additional active agent, whereby the envelope comprises at least one enteric coating, said envelope being adapted for preventing substantially any release of the SSRI compound and the additional active agent in the stomach, said envelope being selected from the group consisting of envelope rendered permeable at pH comprised between 6 and 7.5, envelope disintegrable at pH comprised between 6 and 7.5, envelope solubilized at pH comprised between 6 and 7.5, and combinations thereof and whereby the first SSRI containing form is provided with a delay release means for ensuring a delay release period of at least 20 minutes for the SSRI compound at pH comprised between 6.8 and 7.5 with a rate of release of less than 2 mg SSRI per hour.

28. The unit dosage form of claim 20, which comprises an envelope containing at least the first SSRI containing form and the second form containing the additional active agent, whereby the envelope comprises at least one enteric coating, said envelope being adapted for preventing substantially any release of the SSRI compound and the additional active agent in the stomach, said envelope being selected from the group consisting of envelope rendered permeable at pH comprised between 6 and 7.5, envelope disintegrable at pH comprised between 6 and 7.5, envelope solubilized at pH comprised between 6 and 7.5, and combinations thereof and whereby the first SSRI containing form is provided with a delay release coating adapted for ensuring a delay release period of at least 20 minutes for the SSRI compound at pH comprised between 6.8 and 7.5 with a rate of release of less than 2 mg SSRI per hour.

29. The unit dosage form of claim 20, which comprises an envelope containing at least the first SSRI containing form and the second form containing the additional active agent, whereby the envelope comprises at least one enteric coating, said envelope being adapted for preventing substantially any release of the SSRI compound and the additional active agent in the stomach, said envelope being selected from the group consisting of envelope rendered permeable at pH comprised between 6 and 7.5, envelope disintegrable at pH comprised between 6 and 7.5, envelope solubilized at pH comprised between 6 and 7.5, and combinations thereof and whereby the first SSRI containing form is in the form of SSRI containing pellets provided with a delay release coating adapted for ensuring a delay release period comprised between 20 minutes and 60 minutes for the SSRI compound at pH comprised between 6.8 and 7.5 with a rate of release of less than 2 mg SSRI per hour.

30. The unit dosage form of claim 20, in which the second form containing the additional active agent is a form adapted for ensuring the release of at least 90% of said additional active agent in less than 5 minutes at a pH comprised between 6 and 7.5.

31. The unit dosage form of claim 20, which comprises a water soluble envelope containing at least the first SSRI containing form and the second form containing the additional active agent, whereby the first SSRI containing form is provided with a delay release means adapted for preventing substantially any SSRI release in the stomach, while ensuring a delay release period of at least 10 minutes for the SSRI compound at pH comprised between 6.8 and 7.5 with a rate of release of less than 2 mg SSRI per hour.

32. The unit dosage form of claim 20, which comprises a water soluble envelope containing at least the first SSRI containing form and the second form containing the additional active agent, whereby the first SSRI containing form is provided with a delay release means adapted for preventing substantially any SSRI release in the stomach, while ensuring a delay release period of at least 20 minutes for the SSRI compound at pH comprised between 6.8 and 7.5 with a rate of release of less than 2 mg SSRI per hour.

33. The unit dosage form of claim 20, which comprises a water soluble envelope containing at least the first SSRI containing form and the second form containing the additional active agent, whereby the first SSRI containing form is provided with an enteric coating adapted for preventing substantially any SSRI release in the stomach, while ensuring a delay release period of at least 20 minutes for the SSRI compound at pH comprised between 6.8 and 7.5 with a rate of release of less than 2 mg SSRI per hour.

34. The unit dosage form of claim 20, which comprises a water soluble envelope containing at least the first SSRI containing form and the second form containing the additional active agent, whereby the first SSRI containing form is in the form of SSRI containing pellets provided with an enteric coating adapted for preventing substantially any SSRI release in the stomach, while ensuring a delay release period between 20 minutes and 60 minutes for the SSRI compound at pH comprised between 6.8 and 7.5 with a rate of release of less than 2 mg SSRI per hour.

35. The unit dosage form of claim 20, in which the second form containing the additional active agent is a form adapted for ensuring the release of at least 90% of said additional active agent in less than 5 minutes at a pH comprised between 6 and 7.5.

36. The unit dosage form of claim 20, in which the second form is in the form of pellets containing the additional active agent.

37. The unit dosage form of claim 20, in which the second form containing the additional active agent is a form adapted for preventing substantially any release of the additional active agent in the stomach, while ensuring the release of at least 90% of said additional active agent in less than 5 minutes at a pH comprised between 6 and 7.5.

38. The unit dosage form of claim 20, in which the second form is in the form of pellets containing the additional active agent, whereby said pellets are provided with an enteric coating adapted for preventing substantially any release of the additional active agent in the stomach, while ensuring the release of at least 90% of said additional active agent in less than 5 minutes at a pH comprised between 6 and 7.5.

39. The unit dosage form of claim 20, in which the first SSRI containing form is a SSRI containing form provided with a coating comprising at least an inwards coating layer, an outwards coating layer, and an intermediate coating layer located between the inwards coating layer and the outwards coating layer, whereby the inwards coating layer is located between the SSRI containing core and the outwards coating layer, whereby the intermediate coating layer comprises the additional active agent, while the inwards coating layer is selected from the group consisting of enteric coating layer adapted for preventing substantially any SSRI release in the stomach, while ensuring a delay release period of at least 10 minutes for the SSRI compound at pH comprised between 6.8 and 7.5 with a rate of release of less than 2 mg SSRI per hour, and coating layer ensuring a delay release period of at least 10 minutes for the SSRI compound at pH comprised between 6.8 and 7.5 with a rate of release of less than 2 mg SSRI per hour.

40. The unit dosage form of claim 20, in which the first SSRI containing form is a SSRI containing form provided with a coating comprising at least an inwards coating layer, an outwards coating layer, and an intermediate coating layer located between the inwards coating layer and the outwards coating layer, whereby the inwards coating layer is located between the SSRI containing core and the outwards coating layer, whereby the intermediate coating layer comprises the additional active agent, while the inwards coating layer is selected from the group consisting of enteric coating layer adapted for preventing substantially any SSRI release in the stomach, while ensuring a delay release period of at least 20 minutes for the SSRI compound at pH comprised between 6.8 and 7.5 with a rate of release of less than 2 mg SSRI per hour, and coating layer ensuring a delay release period of at least 20 minutes for the SSRI compound at pH comprised between 6.8 and 7.5 with a rate of release of less than 2 mg SSRI per hour.

41. The unit dosage form of claim 20, in which the first SSRI containing form is a SSRI containing form provided with a coating comprising at least an inwards coating layer, an outwards coating layer, and an intermediate coating layer located between the inwards coating layer and the outwards coating layer, whereby the inwards coating layer is located between the SSRI containing core and the outwards coating layer, whereby the intermediate coating layer comprises the additional active agent, while the outwards coating layer is an enteric coating layer adapted for preventing substantially any SSRI release and any additional active agent release in the stomach, and while the inwards coating layer is adapted for ensuring a delay release period of at least 10 minutes for the SSRI compound at pH comprised between 6.8 and 7.5 with a rate of release of less than 2 mg SSRI per hour.

42. The unit dosage form of claim 20, in which the first SSRI containing form is a SSRI containing form provided with a coating comprising at least an inwards coating layer, an outwards coating layer, and an intermediate coating layer located between the inwards coating layer and the outwards coating layer, whereby the inwards coating layer is located between the SSRI containing core and the outwards coating layer, whereby the intermediate coating layer comprises the additional active agent, while the outwards coating layer is an enteric coating layer adapted for preventing substantially any SSRI release and any additional active agent release in the stomach, and while the inwards coating layer is adapted for ensuring a delay release period of at least 20 minutes for the SSRI compound at pH comprised between 6.8 and 7.5 with a rate of release of less than 2 mg SSRI per hour.

43. A unit dosage form suitable for oral administration to a human, comprising:
a first SSRI containing form comprising an effective antidepressant amount of a SSRI compound selected from the group consisting of fluoxetine, citalopram, escitalopram, paroxetine, venlafaxine, duloxetine, dapoxetine, fluvoxamine, sertraline, and pharmaceutically acceptable salts thereof, at least one pharmaceutically acceptable carrier, and at least one release delay means in an amount adapted, so that, when dissolution tested in vitro in a USP-2 apparatus containing 900 ml of acetate buffer, pH 4.0, which is 0.075 M in NaCl, said USP-2 apparatus being equipped with a paddle stirring at 50 rpm, the SSRI containing form releases the SSRI compound at a rate less than 1 mg SSRI/hr for a delay period comprised between 20 minutes and 4 hours and, thereafter at a rate greater than 1 mg SSRI/hr, and
a second form comprising an effective amount of less than 50 mg of an additional active agent selected from the group consisting of acetyl salicylic acid, pharmaceutically acceptable salts thereof, pharmaceutically acceptable esters thereof, diaspirin and mixtures thereof, whereby said form is adapted, when dissolution tested in vitro in a USP-2 apparatus containing 900 ml of acetate buffer, pH 4.0, which is 0.075 M in NaCl, releases at least 90% by weight of the additional active agent in less than the said delay period.

44. The unit dosage form of claim 43 comprising:
a first SSRI containing form comprising an effective antidepressant amount of a SSRI compound selected from the group consisting of fluoxetine, citalopram, escitalopram, paroxetine, venlafaxine, duloxetine, dapoxetine, fluvoxamine, sertraline, and pharmaceutically acceptable salts thereof, at least one pharmaceutically acceptable carrier, and at least one release delay means in an amount adapted, so that, when dissolution tested in vitro in a USP-2 apparatus containing 900 ml of acetate buffer, pH 4.0, which is 0.075 M in NaCl, said USP-2 apparatus being equipped with a paddle stirring at 50 rpm, the SSRI containing form releases the SSRI compound at a rate less than 1 mg SSRI/hr for a delay period comprised between 20 minutes and 2 hours and, thereafter at a rate greater than 1 mg SSRI/hr, and
a second form comprising an effective amount of less than 50 mg of an additional active agent selected from the group consisting of acetyl salicylic acid, pharmaceutically acceptable salts thereof, pharmaceutically acceptable esters thereof, diaspirin and mixtures thereof, whereby said form is adapted, when dissolution tested in vitro in a USP-2 apparatus containing 900 ml of acetate buffer, pH 4.0, which is 0.075 M in NaCl, releases at least 90% by weight of the additional active agent in less than the said delay period.

45. The unit dosage form of claim 43, in which the first SSRI containing form comprises an effective antidepressant amount of a SSRI compound selected from the group consisting of fluoxetine, citalopram, escitalopram, paroxetine, venlafaxine, duloxetine, dapoxetine, fluvoxamine, sertraline, and pharmaceutically acceptable salts thereof, at least one pharmaceutically acceptable carrier, at least one release delay means, and at least a control sustain release means, whereby said release delay means and control sustain release means are present in an amount adapted, so that, when dissolution tested in vitro in a USP-2 apparatus containing 900 ml of acetate buffer, pH 4.0, which is 0.075 M in NaCl, said USP-2 apparatus being equipped with a paddle stirring at 50 rpm, the SSRI containing form releases the SSRI compound at a rate less than 1 mg SSRI/hr for a delay period comprised between 20 minutes and 4 hours and, thereafter at a rate from 1 mg SSRI/hr to 40 mg SSRI/hr for a period of at least 1 hour after the delay period.

46. The unit dosage form of claim 43, in which the first SSRI containing form comprises an effective antidepressant amount of a SSRI compound selected from the group consisting of fluoxetine, citalopram, escitalopram, paroxetine, venlafaxine, duloxetine, dapoxetine, fluvoxamine, sertraline, and pharmaceutically acceptable salts thereof, at least one pharmaceutically acceptable carrier, at least one release delay means, and at least a control sustain release means, whereby said release delay means and control sustain release means are present in an amount adapted, so that, when dissolution tested in vitro in a USP-2 apparatus containing 900 ml of acetate buffer, pH 4.0, which is 0.075 M in NaCl, said USP-2 apparatus being equipped with a paddle stirring at 50 rpm, the SSRI containing form releases the SSRI compound at a rate less than 1 mg SSRI/hr for a delay period comprised between 20 minutes and 2 hours and, thereafter at a rate from 1 mg SSRI/hr to 40 mg SSRI/hr for a period of at least 2 hours after the delay period.

47. The unit dosage form of claim 43, in which the first SSRI containing form comprises an effective antidepressant amount of a SSRI compound selected from the group consisting of fluoxetine, citalopram, escitalopram, paroxetine, venlafaxine, duloxetine, dapoxetine, fluvoxamine, sertraline, and pharmaceutically acceptable salts thereof, at least one pharmaceutically acceptable carrier, at least one release delay means, and at least a control sustain release means, whereby said release delay means and control sustain release means are present in an amount adapted, so that, when dissolution tested in vitro in a USP-2 apparatus containing 900 ml of acetate buffer, pH 4.0, which is 0.075 M in NaCl, said USP-2 apparatus being equipped with a paddle stirring at 50 rpm, the SSRI containing form releases the SSRI compound at a rate less than 1 mg SSRI/hr for a delay period comprised between 20 minutes and 2 hours and, thereafter at a rate from 1 mg SSRI/hr to 40 mg SSRI/hr for a period of at least 1 hour after the delay period, provided that less than 80% by weight of the SSRI compound is released within said period of 1 hour.

48. The unit dosage form of claim 43, in which the first SSRI containing form comprises a core element selected from the group consisting of matrix tablet, osmotic tablet, hydrogel tablet, multiparticulates and combinations thereof, said core element being provided with an outer coating comprising at least one release delay layer, said outer coating being adapted, so that, when dissolution tested in vitro in a USP-2 apparatus containing 900 ml of acetate buffer, pH 4.0, which is 0.075 M in NaCl, said USP-2 apparatus being equipped with a paddle stirring at 50 rpm, the SSRI containing form releases the SSRI compound at a rate less than 1 mg SSRI/hr for a delay period comprised between 20 minutes and 2 hours and, thereafter at a rate greater than 1 mg SSRI/hr which remains substantially intact during the period of sustained release.

49. The unit dosage form of claim 43, in which the first SSRI containing form comprises a core element selected from the group consisting of matrix tablet, osmotic tablet, hydrogel tablet, multiparticulates and combinations thereof, said core element being placed within a capsule adapted, so that, when dissolution tested in vitro in a USP-2 apparatus containing 900 ml of acetate buffer, pH 4.0, which is 0.075 M in NaCl, said USP-2 apparatus being equipped with a paddle stirring at 50 rpm, the SSRI compound is released at a rate less than 1 mg SSRI/hr for a delay period comprised between 20 minutes and 2 hours and, thereafter at a rate greater than 1 mg SSRI/hr which remains substantially intact during the period of sustained release.

50. The unit dosage form of claim 43, in which the first SSRI containing form comprises a core element selected from the group consisting of matrix tablet, osmotic tablet, hydrogel tablet, multiparticulates and combinations thereof, said core element being provided with an outer coating comprising at least one release delay layer, and being placed within a capsule, so that, when dissolution tested in vitro in a USP-2 apparatus containing 900 ml of acetate buffer, pH 4.0, which is 0.075 M in NaCl, said USP-2 apparatus being equipped with a paddle stirring at 50 rpm, the SSRI compound is released at a rate less than 1 mg SSRI/hr for a delay period comprised between 20 minutes and 2 hours and, thereafter at a rate greater than 1 mg SSRI/hr, which remains substantially intact during the period of sustained release.

51. The unit dosage form of claim 20, in which the second form comprising an effective amount of less than 50 mg of an additional active agent selected from the group consisting of acetyl salicylic acid, pharmaceutically acceptable salts thereof, pharmaceutically acceptable esters thereof, diaspirin and mixtures thereof, is in the form selected from the group consisting of tablets, microtablets, multiparticulates, powders, pellets and combinations thereof.

52. The unit dosage form of claim 51, in which the second form further comprises at least a water soluble pharmaceutically acceptable carrier having a water solubility at 37° C. of more than 5 g per 100 ml water.

53. The unit dosage form of claim 20, in which the second form comprises an effective amount of less than 50 mg of an additional active agent selected from the group consisting of acetyl salicylic acid, pharmaceutically acceptable salts thereof, pharmaceutically acceptable esters thereof, diaspirin and mixtures thereof, and a water soluble pharmaceutically acceptable carrier having a water solubility at 37° C. of more than 25 g per 100 ml water.

54. The unit dosage form of claim 53, in which the water-soluble pharmaceutically acceptable carrier is selected from the group consisting of saccharides and glycine betaine and salts thereof.

55. The unit dosage form of claim 54, comprising less than 300 mg glycine betaine or salt thereof.

56. The unit dosage form of claim 20, in which the first SSRI containing form is selected from the group consisting of solid form, semi solid form and combinations thereof.

57. The unit dosage form of claim 20, in which the second form is selected from the group consisting of solid form, semi solid form and combinations thereof.
